(19) United States
(12) Patent Application Publication
(10) Pub. No.: US 2017/0216399 A1
(43) Pub. Date: Aug. 3, 2017

(54) COMPOSITIONS, METHODS AND USES FOR PROTECTING WHITE BLOOD CELLS FROM SUPPRESSION OR DEATH

(71) Applicant: LOS ANGELES BIOMEDICAL RESEARCH INSTITUTE AT HARBO-UCLA MEDICAL CENTER, Torrance, CA (US)

(72) Inventors: Ronald S. SWERDLOFF, Long Beach, CA (US); YanHe LUE, Torrance, CA (US); Christina WANG, Long Beach, CA (US)

(21) Appl. No.: 15/510,674

(22) PCT Filed: Sep. 14, 2015
(86) PCT No.: PCT/US15/50029

§ 371 Date: Mar. 10, 2017

Related U.S. Application Data
(60) Provisional application No. 62/049,960, filed on Sep. 12, 2014.

(51) Int. Cl. A61K 38/17

(52) U.S. Cl. CPC A61K 38/1761

(57) ABSTRACT

Provided herein are compositions, methods and uses of humanin or a humanin analog, for example, in treating a subject with humanin or a humanin analog, in part, to protect white blood cells from suppression or cell death induced by an autoimmune, anti-tumor or anti-cancer therapeutic agent.

### RELATED APPLICATION INFORMATION

This application is the National Phase of International Application No. PCT/US2015/050029, filed Sep. 14, 2015 which designated the U.S. and that International Application was published under PCT Article 21(2) in English, and claims priority to application Ser. No. 62/049,960, filed Sep. 12, 2014, all of which applications are expressly incorporated herein by reference in their entirety, including all text, tables, sequence listings and drawings.

### SEQUENCE LISTING

The instant application contains a Sequence Listing which has been submitted electronically in ASCII format and is hereby incorporated by reference in its entirety. Said ASCII copy, created on Mar. 10, 2017, is named LABIO0451679_ST25.txt and is 31,333 bytes in size.

### INTRODUCTION

Chemotherapy is the first-line treatment for many cancers in young men including leukemia, lymphoma, testicular tumors, central nervous system tumors, and melanomas. After cycles of combined cancer chemotherapy many young men have long term cancer-free survival but are infertile because chemotherapeutic drugs induced cell death of immature germ cells including spermatogonial stem cells.

Cyclophosphamide (CP) is a chemotherapeutic agent used in men and in experimental animals. CP treatment causes germ cell damage in rodents and men but requires liver cytochrome P450 metabolism to generate an active metabolite, 4-hydroxy-cyclophosphamide, which circulates to cancer cells and damages DNA leading to apoptosis (Sloderbach, et al., 2013). During in vivo treatment with CP, all differentiated germ cells of mice were eliminated and about half of the undifferentiated germ cells remained. Once treatment was stopped, about 64% of germ cells regenerated from these undifferentiated stem cells (Drumond, et al., 2011). Even though CP does not completely eliminate stem spermatogonia in the short term, stem spermatogonia loss may not be completely reversible in cancer patients after multiple cycles of cancer chemotherapy. Some recovery has been reported in rodents (Delbes, et al., 2010) and men (Dohle, 2010; Meistrich, 2013; Trost & Brannigan, 2012). Currently in men, cryopreservation of spermatozoa is the recommended method to preserve male fertility in post-pubertal men and experimental cryopreservation of testicular tissue for prepubertal boys. Semen samples should be collected prior to chemotherapy in couples that desire future fertility. However, if the disease is metastatic and the patient is ill, spermatogenesis may be impaired and the quality of the sample collected before treatment may be poor and not suitable for use in assisted reproductive technologies (Choy & Brannigan, 2013; Loren, et al., 2013; Nangia, et al., 2013).

Humanin (HN), a 24-amino acid mitochondrial derived peptide, is an endogenous anti-apoptotic peptide in many tissues including neurons (Hashimoto, et al., 2001; Hashimoto, et al., 2001), endothelial cells (Bachar, et al., 2010), pancreatic beta cells (Hoang, et al., 2010), and cardiomyocytes (Muzumdar, et al., 2010). HN is expressed in germ cells and Leydig cells in testes (Colon, et al., 2006; Moretti, et al., 2010). I-IN reportedly protects against male germ cell apoptosis induced by testicular hormonal deprivation (Jia, et al., 2013; Lue, et al., 2010). In addition to the finding of endogenous HN (peptide or gene) in normal tissues and cells, HN has been proposed as an potential oncopeptide (Maximov, et al., 2002) because HN gene is expressed in cutaneous T-cell lymphoma (Hartmann, et al., 2008), diffuse large B-cell lymphoma (Tarantul & Hunsmann, 2001), and gastric cancer (Mottaghi-Dastjerdi, et al., 2014).

### SUMMARY

In some aspects presented herein is a method of protecting, reducing, decreasing, or inhibiting white blood cells (WBC) in a subject from an autoimmune, anti-cancer or anti-tumor therapeutic agent suppression or death, where white blood cell suppression or death is induced, promoted, increased, or stimulated by an alkylating agent, comprising administering to a subject prior to, during or after treatment with an autoimmune, anti-cancer or anti-tumor therapeutic agent an amount of humanin or a humanin analog sufficient to protect, reduce, decrease, or inhibit white blood cells in the subject from suppression or death induced, promoted, increased, or stimulated by the autoimmune, anti-cancer or anti-tumor therapeutic agent.

Also presented herein in certain aspects is a method of promoting or increasing maturation, proliferation or survival of white blood cells (WBC) in a subject, wherein white blood cell maturation, proliferation or survival is reduced, decreased, or inhibited by an autoimmune, anti-cancer or anti-tumor therapeutic agent, comprising administering to a subject prior to, during or after treatment with the autoimmune, anti-cancer or anti-tumor therapeutic agent an amount of humanin or a humanin analog sufficient to promote or increase maturation, proliferation or survival of white blood cells in the subject.

Also presented herein in certain aspects is a method of reducing, decreasing, or inhibiting damage to white blood cells (WBC) in a subject by an autoimmune, anti-cancer or anti-tumor therapeutic agent, wherein white blood cells are damaged by an autoimmune, anti-cancer or anti-tumor therapeutic agent, comprising administering to a subject prior to, during or after treatment with the autoimmune, anti-cancer or anti-tumor therapeutic agent an amount of humanin or a humanin analog sufficient to reduce, decrease, or inhibit damage of white blood cells in the subject.

Also presented herein in certain aspects is a method of reducing, decreasing, or inhibiting infection of a subject treated with an autoimmune, anti-cancer or anti-tumor therapeutic agent, comprising administering to a subject prior to, during or after treatment with the autoimmune, anti-cancer or anti-tumor therapeutic agent an amount of humanin or a humanin analog sufficient to reduce, decrease, or inhibit infection of the subject.

Also presented herein in some aspects is a humanin or a humanin analog in the manufacture of a medicament 1) for protecting white blood cells (WBC) from autoimmune, anti-cancer or anti-tumor therapeutic agent suppression or death, 2) for promoting or increasing maturation, proliferation or survival of white blood cells (WBC), where white blood cell maturation, proliferation or survival is reduced, decreased, or inhibited by an autoimmune, anti-cancer or anti-tumor therapeutic agent, 3) to reduce, decrease, or inhibit damage of white blood cells caused by an autoimmune, anti-cancer or anti-tumor therapeutic agent; or 4) to reduce, decrease, or inhibit infection of a subject treated with an autoimmune, anti-cancer or anti-tumor therapeutic agent.

In some aspects of the methods and uses humanin or humanin analog provides protection, reduction, decrease, or inhibition from suppression or death cells of myeloid lineage. In some aspects of the methods and uses protection, reduction, decrease, or inhibition from suppression or death provided by humanin or humanin analog is relatively greater for white blood cells (WBC) of myeloid lineage than of lymphoid lineage. In additional aspects, the protecting, reducing, decreasing, or inhibiting from suppression or death provided by humanin or humanin analog is relatively greater for granulocytes and/or monocytes than for lymphocytes.

In some aspects of the methods and uses a subject has a hyperproliferative or autoimmune disease or disorder. In some aspects of the methods and uses the subject has a metastatic or non-metastatic neoplasia, tumor, cancer or malignancy. In certain aspects of the methods and uses the autoimmune, anti-cancer or anti-tumor therapeutic agent comprises an alkylating agent, an anthracycline, an anti-metabolite, plant extract, plant alkaloid, nitrosourea, hormone, nucleoside or nucleotide analog. In some aspects of the method or use the autoimmune, anti-cancer or anti-tumor therapeutic agent comprises a DNA intercalating agent or an agent that attaches or bonds to DNA.

In some embodiments of the methods and uses the autoimmune, anti-cancer or anti-tumor therapeutic agent comprises cyclophosphamide, acrolein, phosphoramide, doxorubicin, azathioprine, cyclosporin A, prednisolone, melphalan, chlorambucil, mechlorethamine, busulphan, methotrexate, 6-mercaptopurine, thioguanine, 5-fluorouracil, cytosine arabinoside, 5-azacytidine (5-AZC) and 5-azacytidine related compounds, bleomycin, actinomycin D, mithramycin, mitomycin C, carmustine, lomustine, semustine, streptozotocin, hydroxyurea, cisplatin, carboplatin, oxiplatin, mitotane, procarbazine, dacarbazine, a taxane, vinblastine, vincristine, dibromomannitol, gemcitabine, or pemetrexed.

In some aspects the humanin or a humanin analog does not substantially reduce, decrease, suppress or inhibit efficacy or activity of the autoimmune, anti-cancer or anti-tumor therapeutic agent. In some embodiments of the methods and uses the efficacy or activity of the autoimmune, anti-cancer or anti-tumor therapeutic agent comprises partial or complete destruction of a hyperproliferating cell, or a neoplastic, tumor, cancer or malignant cell mass, volume, size or numbers of cells; stimulating, inducing or increasing hyperproliferating cell or neoplastic, tumor, cancer or malignant cell necrosis, lysis or apoptosis; reduces hyperproliferating cell or neoplasia, tumor, cancer or malignancy volume size or cell mass; inhibits or prevents progression or an increase in hyperproliferating cell or neoplasia, tumor, cancer or malignancy volume, mass, size or cell numbers, reduces neoplasia, tumor, cancer or malignancy metastasis volume, size or cell mass; or prolongs lifespan.

In certain embodiments humanin comprises the sequence: MAPRGFSCLLLLTSEIDLPVKRRA.

In certain embodiments humanin analog comprises the sequence: MAPRGFSCLLLLTGEIDLPVKRRA (HN-S14G), or any sequence set forth in Tables 1-5.

In some embodiments, the neoplasia, tumor, cancer or malignancy is metastatic, non-metastatic or benign. In some embodiments, the neoplasia, tumor, cancer or malignancy comprises a solid cellular mass. In some embodiments, the neoplasia, tumor, cancer or malignancy comprises hematopoietic cells. In certain embodiments, the neoplasia, tumor, cancer or malignancy comprises a carcinoma, sarcoma, lymphoma, leukemia, adenoma, adenocarcinoma, melanoma, glioma, glioblastoma, meningioma, neuroblastoma, retinoblastoma, astrocytoma, oligodendrocytoma, mesothelioma, reticuloendothelial, lymphatic or haematopoietic neoplasia, tumor, cancer or malignancy. In some embodiments, the sarcoma comprises a lymphosarcoma, liposarcoma, osteosarcoma, chondrosarcoma, leiomyosarcoma, rhabdomyosarcoma or fibrosarcoma. In some embodiments, the haematopoietic cell neoplasia, tumor, cancer or malignancy comprises a myeloma, lymphoma or leukemia. In some embodiments, the neoplasia, tumor, cancer or malignancy comprises a metastatic melanoma. In some aspects of the methods and uses the neoplasia, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin, lung, biliary tract, or hematologic neoplasia, tumor, or cancer.

In certain embodiments the autoimmune disease or disorder comprises rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, ulcerative colitis (UC), inflammatory bowel disease (IBD), aphthous ulcer, iritis, conjunctivitis, keratoconjunctivitis, cutaneous lupus erythematosus, scleroderma, uveitis posterior, autoimmune uveitis, allergic encephalomyelitis, aplastic anemia, pernicious anemia, pure red cell anemia, idiopathic thrombocytopenic purpura (ITP), polychondritis, Wegener's granulomatosis, Stevens-Johnson syndrome, idiopathic sprue, lichen planus, Graves' disease, sarcoidosis, primary biliary cirrhosis, Hashimoto's thyroiditis, autoimmune polyglandular syndrome, insulin-dependent diabetes mellitus (IDDM, type I diabetes), insulin-resistant diabetes mellitus (type 11 diabetes), autoimmune Addison's disease, pemphigus vulgaris, pemphigus foliaceus, dermatitis herpetiformis, autoimmune alopecia, vitiligo, autoimmune hemolytic anemia, Guillain-Barre syndrome, stiff-man syndrome, acute rheumatic fever, sympathetic ophthalmia, Goodpasture's syndrome, systemic necrotizing vasculitis, antiphospholipid syndrome, Behcet's disease, X-linked lymphoproliferative syndrome (SH2D1A/SAP deficiency), or Graft vs. Host Disease (GVHD).

In some aspects, the infection comprises a bacterial, viral, fungal, or parasite infection. In certain aspects the infection comprises re-activation of latent viruses in the subject.

In some embodiments the methods or uses further comprising administering or use of a second, third or fourth autoimmune, anti-cancer or anti-tumor therapeutic agent. In some embodiments the humanin or humanin analog is administered or used prior to, substantially contemporaneously with or following administration of the autoimmune, anti-cancer or anti-tumor therapeutic agent. In some embodiments the humanin or humanin analog is administered or used in combination with the autoimmune, anti-cancer or anti-tumor therapeutic agent. In some embodiments the humanin or humanin analog is administered or used in one or more dose amounts of 0.05 to 50 mg/Kg per day. In some embodiments humanin or the humanin analog is administered or used in one or more dose amounts of 0.1 to 25 mg/Kg per day, 0.5 to 15 mg/Kg per day, or 1.0 to 10 mg/Kg per day.

In some embodiments of the methods and uses the subject has undergone surgical resection, chemotherapy, immunotherapy, ionizing or chemical radiotherapy, local or regional thermal (hyperthermia) therapy, or vaccination. In some embodiments the subject is or is not a candidate for surgical resection, chemotherapy, immunotherapy, ionizing or chemical radiotherapy, local or regional thermal (hyperthermia) therapy, or vaccination. In some embodiments the subject is a mammal. In some embodiments the subject is a primate. In some embodiments the subject is a human.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIGS. 1A-1B show marked suppression of total white blood cells (FIG. 1A) and granulocytes (FIG. 1B) in mice treated with cyclophosphamide (CP), Human variant S14G-HN (HNG), CP and HNG (CPHNG) or no treatment (Con) for two days. Concomitant treatment with HNG partially rescued the decrease in white blood cells and granulocytes in blood.

FIG. 2 shows that cyclophosphamide (CP) induced suppression of WBC is reversed by HNG concomitant treatment in mice bearing metastatic melanoma in their lungs.

FIGS. 3A-3C show that administration of HNG increased plasma HNG levels in both groups treated with exogenous TING (HNG and HNG+CP groups) as compared to non-treated tumor-bearing (NT) and CP alone group (FIG. 3A). HNG or CP treatment alone significantly suppressed plasma IGF-1. Addition of HNG to CP further suppressed IGF-1 compared to CP alone (FIG. 3B). Co-treatment of HNG with CP increases plasma IGFBP-1 when compared to CP alone (FIG. 3C).

FIGS. 4A-4D show that CP treatment significantly suppressed white blood cell numbers (FIG. 4A), granulocytes (FIG. 4B), monocytes (FIG. 4C) and lymphocytes (FIG. 4D) compared to non-treated tumor-bearing mice. Addition of HNG to CP significantly increased white blood cells (FIG. 4A), granulocytes (FIG. 4B), and monocytes (FIG. 4C) compared to CP treatment alone. Addition of HNG to CP did not significantly rescue the decrease in lymphocytes induced by a single dose of CP (FIG. 4D).

### DETAILED DESCRIPTION

Humanin (HN), a 24-amino acid mitochondrial derived peptide, is an endogenous anti-apoptotic peptide in many tissues. As disclosed herein, HN and HN analogs can be used as agents to protect white blood cells suppression, death and/or damage caused by therapeutic agents used for treatment of autoimmune, cancer and tumors. For example, HN and HN analogs were able to protect animals against chemotherapy induced leucopenia (neutropenia) and limit/protect the suppression of white blood cells caused by cyclophosphamide treatment. These discoveries are clinically relevant, as methods, uses and compositions described herein can be used as an adjunct to treatments of autoimmune and hyperproliferative diseases and disorders, (e.g., neoplasias, tumors, cancers and malignancies) in which white blood cells are suppressed or killed by treatment with chemotherapy.

In certain embodiments, method and compositions herein can protect cancer patient from many common adverse acute effects of chemotherapy. For example, method and compositions herein can protect a subject from treatment-induced suppression of white blood cells, which can increase the risk of infections (bacterial, viral, fungal, parasitic, etc.), sepsis, re-activation of latent viruses and increased morbidity and mortality

Methods, uses and compositions herein are applicable to any subject. A subject is any living or non-living organism, including but not limited to a mammal such as a human. A subject can also be a non-human animal, non-limiting examples of which include a reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), primate (e.g., monkey, ape, chimpanzee), ursid (e.g., bear), bird (e.g., poultry, fowl), dog, cat, mouse, rat, fish, dolphin, whale and shark. A subject may be any age (e.g., an embryo, a fetus, infant, child, adult). A subject can be of any sex (e.g., male, female, or combination thereof). A subject may be pregnant. In particular embodiments a subject is a human. In some embodiments a subject is a human patient. A patient can be any subject suspected of having, diagnosed with, or undergoing treatment for an ailment, disease or infection, or a subject who could benefit from a use or method herein. For example, in certain embodiments a patient is a subject diagnosed with a cancer and/or undergoing a treatment for a cancer. In some embodiments a patient is a subject that is immunodeficient, for example due to an immune-suppressive therapy or treatment.

A patient or subject often comprises white blood cells. A white blood cell can be any nucleated immune cell found in the blood, tissue or bone marrow of an animal (e.g., a mammal, a human). Non-limiting examples include granulocytes (e.g., neutrophils, eosinophils, basophils and the like), lymphocytes (e.g., NK cells, NKT cells, T cells, B cells and the like), monocytes, macrophages, dendritic cells, mast cells, microglial cells, the like or combinations thereof. A white blood cell can be any type or sub-type of T cell, non-limiting examples of which include CD4+ T-cell, CD8+ T-cells, CD4− T-cell, CD8− T-cells, Treg cells, naïve T-cells, T-helper cells, cytotoxic T cells, γδ T cells, the like or combinations thereof. White blood cells may be primary cells, immortalized cells and/or transgenic cells. White blood cells can include activated cells, memory cells, senescent cells, tolerant cells, clonal cells, and/or dividing cells. White cells may comprise immune cells at any developmental stage (e.g., stem cells, precursor cells, undifferentiated, differentiated and/or mature cells).

Accordingly, in some embodiments, humanin or humanin analog protection, reduction, decrease, or inhibition from suppression or death can be provided to cells of myeloid lineage. In some embodiments, humanin or humanin analog protection, reduction, decrease, or inhibition from suppression or death can be relatively greater for white blood cells (WBC) of myeloid lineage than of lymphoid lineage. In some embodiments, humanin or humanin analog protection, reduction, decrease, or inhibition from suppression or death can be relatively greater for granulocytes and/or monocytes than for lymphocytes.

In some embodiments, suppression and grammatical variations thereof mean an adverse effect of a therapeutic agent, pathogenic agent or mechanical activity on a white blood cell that results in the inhibition, reduction or loss of one or more functions of the white blood cell. In some embodiments the inhibition, reduction or loss of one or more cell functions refers to the loss of, or inhibition of, a cell's ability to replicate (e.g., proliferate) and/or undergo mitosis or meiosis. In some embodiments the inhibition, reduction or loss of one or more cell functions refers to the loss of, or inhibition of, a cell's ability to metabolize oxygen, proteins, fatty acids, carbohydrates and/or glucose. In some embodiments the inhibition, reduction or loss of one or more cell functions refers to the loss of, or inhibition of, a cell's ability to initiate, carry out, maintain or terminate an adaptive or innate immune response, or a portion thereof. In some embodiments the inhibition, reduction or loss of one or more cell functions refers to the loss of, or inhibition of, a cell's ability to initiate, carry out, maintain or terminate an immune function, non-limiting examples of which include antigen presentation; apoptosis; phagocytosis; pinocytosis; T-cell activation; B-cell activation; expressing, presenting, secreting and/or responding to an antigen, cytokine, chemokine, growth factors, TNF or TNF-related family members, interferon, porin, defensin, complement, protease, antibody, a hormone, and/or receptors thereof; the like; or combinations thereof.

In some embodiments, a therapeutic agent, pathogenic agent or mechanical activity induces cell death or apoptosis of a white blood cell. Cell death can be any type of cell death that is induced by any known or unknown mechanism. In some embodiments cell death refers to apoptotic death (e.g., apoptosis), autophagic cell death (autophagy) and/or necrotic cell death (e.g., necrosis). In some embodiments cell death refers to a loss of cell viability. Cell death and/or viability can be determined by a suitable assay known in the art or described herein, non-limiting examples of which include a suitable membrane alteration assay (e.g., as measured by annexin-V binding, uptake of impermeable dyes such as propidium iodide, trypan blue, LDH release, the like or combinations thereof), caspase activation assays (e.g., as measured by peptide substrate cleavage, substrate cleavage (e.g., PARP, M30), caspase processing, the like or combinations thereof), DNA fragmentation assays (e.g., TUNEL assay, or assessment of DNA laddering, cytoplasmic nucleosomes, hypodipoloid DNA, and release of incorporated nucleotides (e.g., BrdU), the like, or combinations thereof), mitochondrial damage assays (e.g., measurements of cytochrome C release, mitochondrial membrane potential, ATP production, electron transport activity (e.g., WST-1 or MTT assays)), the like or combinations thereof.

Suppression and/or death of white blood cells can be induced by a therapeutic agent. Suppression and/or death of white blood cells can be induced when a white blood cell comes into contact with one or more therapeutic agents. In some embodiments a therapeutic agent is cytotoxic to a white blood cell. In certain embodiments, administration of a therapeutic agent to a subject induces, promotes, increases and/or stimulates suppression and/or death of white blood cells in the absence of a method described herein (e.g., in the absence of administering humanin or a humanin analog). In certain embodiments, administration of a therapeutic agent to a subject reduces, decreases, or inhibits maturation, proliferation and/or survival of white blood cells in the absence of a method described herein (e.g., in the absence of administering humanin or a humanin analog). In certain embodiments, administration of a therapeutic agent to a subject damages white blood cells in the absence of a method described herein (e.g., in the absence of administering humanin or a humanin analog). Cell damage may include damage to genomic DNA, mitochondria or other organelles, mitochondrial DNA, mitochondrial cell walls or phospholipid membranes.

Therapeutic agents can include a variety of poisons, venoms, toxins, proteins, antibodies and inhibitors that can induce suppression and/or death of a white blood cell by a variety of mechanisms. In some embodiments a therapeutic agent comprises a cytotoxic compound. Cytotoxic compound can induce cell death of white blood cells, damage white blood cells and/or inhibit one or more functions of white blood cells. Cytotoxic compounds can be organic compounds. In some embodiments cytotoxic compounds are small organic compounds with a molecular weight between 1 and about 5000 Daltons, 1 and about 2500 Daltons, 1 and about 1000 Daltons, 1 and about 500 Daltons or between about 50 and about 1000 Daltons. Therapeutic agents can be monoclonal or polyclonal antibodies. Therapeutic agents can be polypeptides or fusion proteins. In some embodiments, therapeutic agents are not cytotoxic until after they are administered to a subject wherein the therapeutic agents are metabolized into a cytotoxic compound (e.g., cyclophosphamide). In some embodiments a white blood cell is contacted with a therapeutic agent and the white blood cell metabolizes the therapeutic agent into a cytotoxic compound. White blood cells can be contacted directly or indirectly (e.g., by a targeted approach) with a therapeutic agent. Therapeutic agents can be autoimmune therapeutic agents, anti-cancer therapeutic agents and/or anti-tumor therapeutic agents.

Anti-cancer and/or anti-tumor therapeutic agents are often administered to a subject (e.g., a patient) for the treatment of a hyperproliferative disease or disorder. In some embodiments anti-cancer and/or anti-tumor therapeutic agents comprise or consist of one or more cytotoxic compounds. In some embodiments a therapeutic agent comprises a suitable chemotherapeutic agent. In some embodiments a therapeutic agent comprises or consists of an alkylating agent, an anthracycline, cytoskeletal disruptors, epothilones (e.g., epothilone), histone deacetylase inhibitors (e.g., vorinostat, romidepsin), inhibitors of topoisomerase I (e.g., irinotecan, topotecan), inhibitors of topoisomerase II (e.g., etoposide, teniposide, tafluposidean), kinase inhibitors, peptide antibiotics (e.g., bleomycin, actinomycin), platinum-based agents (e.g., carboplatin, cisplatin, oxaliplatin), retinoids (e.g., tretinoin, alitretinoin, bexarotene), vinca alkaloids and derivatives (e.g., vinblastine, vincristine, vindesine, vinorelbine), anti-metabolites, plant extracts, plant alkaloids, nitrosourea, hormone, nucleoside or nucleotide analog and combinations thereof. Non-limiting examples of alkylating agents include cyclophosphamide, mechlorethamine, chlorambucil, melphalan, monofunctional alkylators, nitrosoureas, temozolomide, the like, known analogs and derivatives thereof. Non-limiting examples of alkylating agents further include anthracyclines, which include daunorubicin, doxorubicin, epirubicin, idarubicin, mitoxantrone, valrubicin, the like, known analogs and derivatives thereof. Non-limiting examples of cytoskeletal disruptors (e.g., taxanes) include paclitaxel, taxol, and docetaxel. Non-limiting examples of kinase inhibitors include bortezomib, erlotinib, gefitinib, imatinib, vemurafenib, vismodegib, the like, known analogs and derivatives thereof. Non-limiting examples of nucleotide analogs include azacitidine, azathioprine, capecitabine, cytarabine, doxifluridine, fluorouracil, gemcitabine, hydroxyurea, mercaptopurine, methotrexate, tioguanine (formerly thioguanine), the like, known analogs and derivatives thereof.

In some embodiments a therapeutic agent comprises a DNA intercalating agent or an agent that attaches or bonds to DNA. In some embodiments a therapeutic agent comprises or consists of cyclophosphamide, acrolein, phosphoramide, doxorubicin, azathioprine, cyclosporin A, prednisolone, melphalan, chlorambucil, mechlorethamine, busulphan, methotrexate, 6-mercaptopurine, thioguanine, 5-fluorouracil, cytosine arabinoside, 5-azacytidine (5-AZC) and 5-azacytidine related compounds, bleomycin, actinomycin D, mithramycin, mitomycin C, carmustine, lomustine, semustine, streptozotocin, hydroxyurea, cisplatin, carboplatin, oxiplatin, mitotane, procarbazine, dacarbazine, a taxane, vinblastine, vincristine, dibromomannitol, gemcitabine, pemetrexed, the like or a combination thereof.

In some embodiments a therapeutic agent (e.g., a chemotherapeutic agent, a cytotoxic compound) induces partial or complete destruction of some or all hyperproliferating cells in a subject. In some embodiments a therapeutic agent induces partial or complete destruction of a neoplastic, tumor, cancer or malignant cell mass in a subject. A therapeutic agent can decrease the volume or size of a neoplasia, neoplastic tumor, cancer or malignancy and/or reduce the numbers of hyperproliferating cells in a subject. In some embodiments a therapeutic agent stimulates and/or induces apoptosis, necrosis, and/or lysis of hyperproliferating cells or cells of a neoplastic tumor, cancer or malignant cell masses in a subject. In some embodiments a therapeutic agent inhibits or prevents progression of or an increase in hyperproliferating cells or a neoplasia, tumor, cancer or malignancy. In some embodiments a therapeutic agent prolongs lifespan of a subject comprising a hyperproliferating disease or disorder. The efficacy or activity of a therapeutic agent can be determined according to 1) its ability and effectiveness to induce partial or complete destruction of some or all hyperproliferating cells in a subject, 2) induce partial or complete destruction of a neoplastic, tumor, cancer or malignant cell mass in a subject, 3) decrease the volume or size of a neoplasia, neoplastic tumor, cancer or malignancy and/or reduce the numbers of hyperproliferating cells in a subject, 4) stimulate and/or induces apoptosis, necrosis, and/or lysis of hyperproliferating cells or cells of a neoplastic tumor, cancer or malignant cell masses in a subject, 5) inhibit or prevent progression of or an increase in hyperproliferating cells or a neoplasia, tumor, cancer or malignancy in a subject, and/or 6) prolong the lifespan of a subject comprising a hyperproliferating disease or disorder. In certain embodiments, the administration of humanin or a humanin analog does not substantially reduce, decrease, suppress or inhibit efficacy or activity of an autoimmune, anti-cancer or anti-tumor therapeutic agent.

Autoimmune therapeutic agents are often administered to a subject (e.g., a patient) for the treatment of an autoimmune disease or disorder. In some embodiments autoimmune therapeutic agents comprise or consist of one or more anti-cancer and/or anti-tumor therapeutic agents. In some embodiments autoimmune therapeutic agents comprise or consist of one or more cytotoxic compounds. In certain embodiments an autoimmune therapeutic agent comprises a folic acid analogue (e.g., methotrexate), azathioprine, mercaptopurine, an anti-CD3 antibody (e.g., muromonab-CD3), an anti-IL2 receptor antibody, ciclosporin (cyclosporine), myriocin, tacrolimus, sirolimus, IFN-β, TNF binding proteins (e.g., infliximab (Remicade), etanercept (Enbrel), and/or adalimumab (Humira)), mycophenolic acid, fingolimod, the like or combinations thereof.

Suppression and/or death of white blood cells can be induced by pathogenic agent, for example by infection with HIV (e.g., HIV1 or HIV2). In certain embodiments, compositions, uses and methods described herein can be used to treat a subject suspected of having, diagnosed with or in remission from an HIV infection.

Suppression and/or death of white blood cells can also be induced by mechanical activity applied directly or indirectly to white blood cells. A mechanical activity applied indirectly to white blood cells can be applied to an organ or tissue comprising white blood cells. Mechanical activities that can induce suppression and/or death of white blood cells can include ionizing treatments such as radiation therapies, non-limiting examples of which include external-beam radiation therapy (e.g., 3-dimensional conformal radiation therapy (3D-CRT), intensity-modulated radiation therapy (IMRT), image-guided radiation therapy (IGRT), tomotherapy, stereotactic radiosurgery, stereotactic body radiation therapy, and proton therapy), internal radiation therapy, systemic radiation therapy, the like or combinations thereof. In some embodiments mechanical activities that induce suppression and/or death of white blood cells can include trauma (e.g., physical trauma (e.g., damage or injury to the spine, bones, brain, spleen and/or thymus)), and/or surgical procedures (e.g., surgical resection, e.g., removal of bone marrow, spleen, thymus and/or portions thereof).

In certain embodiments a method, use or composition herein protects white blood cells from suppression and/or death. In various embodiments, protection of white blood cell suppression and/or death is from a therapeutic agent, pathogenic agent and/or mechanical activity that induces or increases white blood cell suppression and/or death.

In some embodiments compositions, uses and methods herein are used to treat subjects suspected of having, diagnosed with and/or being treated for a hyperproliferative disease or disorder. In some embodiments a hyperproliferative disease or disorder refers to a neoplasia, tumor, cancer or malignancy. In some embodiments a hyperproliferative disease or disorder refers to a subject having a neoplasia, tumor, cancer or malignancy. A hyperproliferative disease or disorder can be metastatic, non-metastatic or benign. In some embodiments a neoplasia, tumor, cancer or malignancy comprises a solid cellular mass. In certain embodiments a malignant neoplasm comprises or consist of a fibrosarcoma, myxosarcoma, liposarcoma, chondrosarcoma, osteosarcoma, chordoma, malignant fibrous histiocytoma, hemangiosarcoma, angiosarcoma, lymphangiosarcoma, mesothelioma, leiomyosarcoma, rhabdomyosarcoma, squamous cell carcinoma, epidermoid carcinoma, malignant skin adnexal tumor, adenocarcinoma, hepatoma, hepatocellular carcinoma, renal cell carcinoma, hypernephroma, cholangiocarcinoma, transitional cell carcinoma, choriocarcinoma, seminoma, embryonal cell carcinoma, glioma, glioblastoma multiforme, neuroblastoma, medulloblastoma, malignant meningioma, malignant schwannoma, neurofibrosarcoma, parathyroid carcinoma, medullary carcinoma of thyroid, bronchial carcinoid, oat cell carcinoma, malignant pheochromocytoma, islet cell carcinoma, malignant carcinoid, retinoblastoma, chemodectoma, paraganglioma, malignant carcinoid, malignant paraganglioma, melanoma, malignant schwannoma, merkel cell neoplasm, cystosarcoma phylloides, wilms tumor, malignant ovarian tumors, malignant testicular tumors, the like, or combinations thereof. In certain embodiments a neoplasia, tumor, cancer or malignancy comprises a carcinoma, sarcoma, lymphoma, leukemia, adenoma, adenocarcinoma, melanoma, glioma, glioblastoma, Kaposi sarcoma, meningioma, neuroblastoma, retinoblastoma, astrocytoma, oligodendrocytoma, reticuloendothelial, lymphatic or haematopoietic neoplasia, tumor, cancer or malignancy. In certain embodiments a sarcoma comprises a lymphosarcoma, liposarcoma, osteosarcoma, chondrosarcoma, leiomyosarcoma, rhabdomyosarcoma or fibrosarcoma.

In some embodiments a hyperproliferative disease or disorder comprises hyperproliferative hematopoietic cells or a haematopoietic cell neoplasia. In some embodiments a haematopoietic cell neoplasia, tumor, cancer or malignancy comprises a myeloma, lymphoma or leukemia. In some embodiments a leukemia is an acute lymphocytic leukemia (ALL), acute myeloid leukemia (AML), chronic lymphocytic leukemia (CLL), chronic myeloid leukemia (CML), or chronic myelomonocytic leukemia (CMML). In certain embodiments a neoplasia, tumor, cancer or malignancy comprises a metastatic melanoma. In certain embodiments a neoplasia, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin, lung, biliary tract, or hematologic neoplasia, tumor, or cancer.

In some embodiments an autoimmune disease or disorder comprises rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, ulcerative colitis (UC), inflammatory bowel disease (IBD), aphthous ulcer, iritis, conjunctivitis, keratoconjunctivitis, cutaneous lupus erythematosus, scleroderma, uveitis posterior, autoimmune uveitis, allergic encephalomyelitis, aplastic anemia, pernicious anemia, pure red cell anemia, idiopathic thrombocytopenic purpura (ITP), polychondritis, Wegener's granulomatosis, Stevens-Johnson syndrome, idiopathic sprue, lichen planus, Graves' disease, sarcoidosis, primary biliary cirrhosis, Hashimoto's thyroiditis, autoimmune polyglandular syndrome, insulin-dependent diabetes mellitus (1DDM, type I diabetes), insulin-resistant diabetes mellitus (type 11 diabetes), autoimmune Addison's disease, pemphigus vulgaris, pemphigus foliaceus, dermatitis herpetiformis, autoimmune alopecia, vitiligo, autoimmune hemolytic anemia, Guillain-Barre syndrome, stiff-man syndrome, acute rheumatic fever, sympathetic ophthalmia, Goodpasture's syndrome, systemic necrotizing vasculitis, antiphospholipid syndrome, Behcet's disease, X-linked lymphoproliferative syndrome (SH2D1A/SAP deficiency), or Graft vs. Host Disease (GVHD).

In some embodiments a composition, use or method herein is used to prevent, reduce, decrease and/or inhibit infection (e.g., a pathogenic infection) in a subject treated with an autoimmune, anti-cancer or anti-tumor therapeutic agent. In certain embodiments a composition, use or method herein is used to prevent, reduce, decrease and/or inhibit infection in an immunosuppressed subject. Therapeutic treatments that are used to treat a subject with an autoimmune, anti-cancer or anti-tumor disease or disorder often suppress or inhibit a treated patient's immune system, which in turn can increase the risk of a subject to infection. Without being limited to theory, certain therapeutic treatments can induce suppression or death of white blood cells thereby rendering a patient's immune system suppressed or inhibited and/or rendering a patient immune-deficient. The administration of a therapeutic agent can increase the probability of infection in a subject several fold. Methods, uses and compositions herein can be used to prevent, reduce, decrease and/or inhibit infection, non-limiting examples of which include bacterial infection, viral infection, fungal infection, or parasite infection, or sepsis. Non-limiting examples of infections include infections from Streptococcus pneumonia, Hemophilus influenza, Pneumocystis jirovecii, Giardia intestinalis, Cryptosporidium parvum, herpes simplex virus, mycobacterium, listeria, intracellular fungal infections, enterobacteriaceae, oral streptococci, Pseudomonas aeruginosa, Enterococcus species, Candida species, Aspergillus species, Neisseria meningitides, Plasmodium species, Babesia species, or combinations thereof. In some embodiments an infection comprises re-activation of latent viruses in a subject.

In some embodiments methods, uses and compositions described herein are to treat a subject having undergone surgical resection, chemotherapy, immunotherapy, ionizing or chemical radiotherapy, local or regional thermal (hyperthermia) therapy, or vaccination. In some embodiments methods, uses and compositions described herein are not used to treat a subject having undergone surgical resection, chemotherapy, immunotherapy, ionizing or chemical radiotherapy, local or regional thermal (hyperthermia) therapy, or vaccination. In certain embodiments methods, uses and compositions described herein are not used to treat a subject that is a candidate for surgical resection, chemotherapy, immunotherapy, ionizing or chemical radiotherapy, local or regional thermal (hyperthermia) therapy, or vaccination.

In some embodiments a method, use or composition described herein protects white blood cells in a subject from suppression and/or death. In some embodiments a method, use or composition described herein protects white blood cells in a subject from suppression and/or death induced by a therapeutic agent, pathogenic agent and/or mechanical activity. Without being limited by theory, a method, use or composition described herein may protect white cells by preserving viability and/or function of one or more white blood cells from the deleterious effects (e.g., adverse effects) caused by administration of a therapeutic agent (e.g., a cytotoxic compound). The term “protect” can mean to prevent, shelter, shield and/or insulate.

In some embodiments a method, use or composition described herein inhibits white blood suppression and/or death. In some embodiments a method, use or composition described herein inhibits white blood cell suppression or death induced by a therapeutic agent, pathogenic agent and/or mechanical activity. Without being limited by theory, a method, use or composition described herein may inhibit white blood cell necrosis, autophagy or apoptosis induced by administration of a therapeutic agent (e.g., a cytotoxic compound). In certain embodiments a method, use or composition described herein may inhibit certain signaling pathways that may lead to apoptosis where the apoptotic pathway is activated by a therapeutic agent. In certain embodiments a method, use or composition described herein may inhibit white blood cell tolerance or senescence.

In some embodiments a method, use or composition described herein reduces white blood cell suppression and/or death. In some embodiments a method, use or composition described herein reduces white blood cell suppression or death induced by a therapeutic agent, pathogenic agent and/or mechanical activity. In some embodiments a method or composition described herein reduces white blood cell death induced or promoted by a therapeutic agent by at least 200%, at least 150%, at least 100%, at least 50%, at least 30%, at least 20%, at least 15%, at least 10%, or at least 5%. In certain embodiments a method, use or composition described herein reduces or inhibits white blood cell suppression (e.g., suppression of proliferation) induced or promoted by a therapeutic agent by at least 200%, at least 150%, at least 100%, at least 50%, at least 30%, at least 20%, at least 15%, at least 10%, or by at least 5%.

In some embodiments a method, use or composition described herein decreases white blood suppression and/or death. In some embodiments a method, use or composition described herein decreases white blood cell suppression or death induced by a therapeutic agent. In some embodiments a method, use or composition described herein decreases white blood cell death induced by a therapeutic agent by up to 100%, up to 50%, up to 30%, up to 20%, up to 15%, up to 10%, or up to 5%. In certain embodiments a method, use or composition described herein decreases white blood cell suppression (e.g., suppression of proliferation) induced by a therapeutic agent by at least 200%, at least 150%, at least 100%, at least 50%, at least 30%, at least 20%, at least 15%, at least 10%, or by at least 5%.

In some embodiments a method, use or composition described herein promotes and/or increases maturation, proliferation and/or survival of white blood cells in a subject. In some embodiments a method, use or composition described herein promotes and/or increases maturation, proliferation and/or survival of white blood cells in a subject that was administered a therapeutic agent. In some embodiments administration or delivery of humanin or a humanin analog promotes and/or increases maturation, proliferation and/or survival of white blood cells in a subject that was administered a therapeutic agent (e.g., a cytotoxic compound), pathogenic agent and/or mechanical activity. In certain embodiments, administration of a therapeutic agent (e.g., a cytotoxic compound) to a subject reduces, decreases, or inhibits maturation, proliferation and/or survival of white blood cells in the absence of administration or delivery of humanin or humanin analog, which reduction, decrease or inhibition can be reversed completely or partially by administration of humanin or a humanin analog. In some embodiments a method, use or composition described herein promote and/or increase maturation, proliferation and/or survival of white blood cells by up to 200%, up to 100%, up to 50%, up to 30%, up to 20%, up to 15%, up to 10%, or up to 5%.

In some embodiments a method, use or composition described herein reduces, decreases or inhibits damage to white blood cells in a subject. In some embodiments a method, use or composition described herein reduces, decreases or inhibits damage to white blood cells in a subject that was administered or delivered a therapeutic agent, pathogenic agent and/or mechanical activity. In some embodiments administration or delivery of humanin or a humanin analog reduces, decreases or inhibits damage to white blood cells in a subject that was administered a therapeutic agent (e.g., a cytotoxic compound), pathogenic agent and/or mechanical activity. In some embodiments a method, use or composition described herein reduces, decreases or inhibits damage to the genomic DNA, mitochondrial DNA, mitochondria, cell organelles or cell membranes of white blood cells in a subject that was administered or delivered a therapeutic agent, pathogenic agent and/or mechanical activity.

In certain embodiments, a method, use or composition herein comprises humanin or a humanin analog. In certain embodiments, a method or use includes administering or delivery of humanin or a humanin analog to subject. Humanin or a humanin analog can be administered or delivered to a subject prior to, during or after administration of a therapeutic agent. Humanin or a humanin analog can be administered to a subject prior to, during or after treatment with a therapeutic agent, pathogenic agent and/or mechanical activity. In some embodiments humanin or a humanin analog is administered or delivered to a subject prior to, during or after administration of an autoimmune, anti-cancer or anti-tumor therapeutic agent. In certain embodiments humanin or a humanin analog is administered or used prior to, substantially contemporaneously with or following administration of an autoimmune, anti-cancer or anti-tumor therapeutic agent. In certain embodiments humanin or a humanin analog is administered or used in combination with an autoimmune, anti-cancer or anti-tumor therapeutic agent.

In some embodiments, human comprises the amino acid sequence of SEQ ID NO: 1. A humanin analog can be a humanin variant. Exemplary non-limiting examples of humanin analogs and/or variants applicable to the methods, uses and compositions set forth herein are shown and described in Tables 1 to 5.

In some embodiments humanin or a humanin analog is administered or delivered in an amount sufficient to protect white blood cells from the adverse effect caused by the presence of a therapeutic agent, pathogenic agent and/or mechanical activity. In some embodiments humanin or a humanin analog is administered or delivered in an amount sufficient to protect, reduce, decrease, or inhibit white blood cells in the subject from suppression or death induced, promoted, increased, or stimulated by an autoimmune, anti-cancer or anti-tumor therapeutic agent. In some embodiments humanin or a humanin analog is administered or delivered in an amount sufficient to promote or increase maturation, proliferation or survival of white blood cells in a subject (e.g., a subject treated with an autoimmune, anti-cancer or anti-tumor therapeutic agent). In some embodiments humanin or a humanin analog is administered or delivered in an amount sufficient to increase white blood cell counts in a subject (e.g., a subject treated with an autoimmune, anti-cancer or anti-tumor therapeutic agent). Methods of determining white blood cell counts are known in the art and any suitable method of determining white blood cell counts can be used.

In some embodiments humanin or a humanin analog is administered or delivered in an amount sufficient to reduce, decrease, or inhibit damage of white blood cells in a subject (e.g., a subject treated with an autoimmune, anti-cancer or anti-tumor therapeutic agent). In some embodiments humanin or a humanin analog is administered or delivered in an amount sufficient to reduce, decrease, or inhibit infection in a subject (e.g., a subject treated with an autoimmune, anti-cancer or anti-tumor therapeutic agent).

As disclosed herein, compositions, methods and uses of the invention, can be administered or delivered prior to, contemporaneously with or after a therapeutic agent, pathogenic agent and/or mechanical activity is administered or delivered, for example to a subject. Accordingly, methods, uses and compositions of the invention can be delivered prior to suppression or death of white blood cells in order to protect white blood cells.

“Prophylaxis” and grammatical variations thereof mean a method in which contact, administration or in vivo delivery to a subject is prior to administration or delivery of a therapeutic agent, pathogenic agent and/or mechanical activity, or prior to white blood cell suppression or death. Administration or in vivo delivery to a subject can therefore be performed prior to onset or detection of white blood cell suppression or death. Accordingly, subjects are candidates for invention compositions, methods and uses, but the subject may not yet exhibit white blood cell suppression or death.

Compositions, methods and uses, such as treatment methods and uses, can provide a detectable or measurable increase in white blood cell counts, improved white blood cell viability (e.g., decreased apoptosis or cell death), and/or white blood cell function in a subject. Compositions, methods and uses of the invention therefore include providing a therapeutic benefit or improvement to a subject, for example, as reflected by increased, stabilized or a less profound reduction in white blood cell counts induced or promoted by a therapeutic agent, pathogenic agent and/or mechanical activity.

A therapeutic benefit or improvement can be any measurable or detectable, objective or subjective, transient, temporary, or longer-term benefit to a subject or improvement in the response, disorder or disease, or one or more adverse symptoms, disorders, illnesses, pathologies, diseases, or complications caused by treatment with a therapeutic agent (e.g., cytotoxic compound or chemotherapeutic agent) and/or associated with a disorder or disease, such as an infection Therapeutic benefits and improvements include, but are not limited to, decreasing, reducing, inhibiting, suppressing, limiting or controlling the occurrence, frequency, severity, progression, or duration of an adverse symptom of undesirable or aberrant response, disorder or disease, such as an infection.

Compositions, methods and uses of the invention, can be administered or delivered in a sufficient or effective amount to a subject. An “effective amount” or “sufficient amount” refers to an amount that provides, in single or multiple doses, alone or in combination, with one or more other compositions (e.g., therapeutic agents or drugs), treatments, protocols, or therapeutic regimens, a detectable response of any duration of time (long or short term), an expected or desired outcome in or a benefit to a subject of any measurable or detectable degree or for any duration of time (e.g., for minutes, hours, days, months, years, or cured).

The doses of a “sufficient amount” for treatment (e.g., to provide a therapeutic benefit or improvement) typically are effective to provide a response (e.g., a measurable increase, stabilization or a reduction white blood cell response). In some embodiments a sufficient amount humanin or a humanin analog comprises an amount between about 0.01 to 100 mg/Kg (mg of humanin or a humanin analog per Kg of a subjects body weight) per day, between about 0.05 to 50 mg/Kg per day, between about 0.1 to 25 mg/Kg per day, between about 0.5 to 15 mg/Kg per day, between about 0.5 to 15 mg/Kg per day, or between about 1.0 to 10 mg/Kg per day. In some embodiments administering a sufficient amount of humanin or a humanin analog comprises administered one or more dose amounts of between about 0.01 to 100 mg/Kg per day, between about 0.05 to 50 mg/Kg per day, between about 0.1 to 25 mg/Kg per day, between about 0.5 to 15 mg/Kg per day, between about 0.5 to 15 mg/Kg per day, or between about 1.0 to 10 mg/Kg per day. A sufficient amount of humanin or a humanin analog may be administered in 1, 2, 3, 4, 5, 6, or 7 doses per day. In some embodiments a sufficient amount of humanin or a humanin analog is administered continuously or intermittently by a patch or suitable device (e.g., a pump). A sufficient amount of humanin or a humanin analog may be self-administered by a subject. For example a subject may use, in one or more doses, a sufficient amount of humanin or a humanin analog.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the response, disorder, or disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol.

As is typical for treatment methods and uses, some subjects will exhibit a greater response, or less or no response to a given treatment method or use. An effective amount or a sufficient amount therefore need not be effective in each and every subject treated, prophylactically or therapeutically, nor a majority of treated subjects in a given group or population. An effective amount or a sufficient amount means effectiveness or sufficiency in a particular subject, not a group or the general population. Accordingly, appropriate amounts will depend upon the condition treated, the therapeutic effect desired, as well as the individual subject (e.g., the bioavailability within the subject, gender, age, etc.).

Effectiveness of a method or use, such as a method of treatment herein can provide a potential therapeutic benefit or improvement that can be ascertained by various methods. Such methods include, for example, measuring white blood cell counts or viability, white blood cell maturation or a response or activity of white blood cells. Measuring T or B cell activation and/or differentiation, cell infiltration of a region, cell accumulation or migration to a region, production of antibodies, cytokines, lymphokines, chemokines, interferons and interleukins, cell growth and maturation factors using various immunological assays, such as ELISA, also can be used to ascertain effectiveness of a method, use or composition as set forth herein.

Humanin and/or humanin analogs, including in combinations with other agents or drugs, can be packaged in a suitable pharmaceutical formulation and/or dosage unit form for ease of administration and uniformity of dosage. “Dosage unit form” as used herein refers to physically discrete units suited as unitary dosages; each unit contains a quantity of the composition optionally in association with a carrier, excipient, diluent, or vehicle calculated to produce the desired treatment or therapeutic (e.g., beneficial) effect. The unit dosage forms can be varied according to factors including, but not necessarily limited to, the particular composition employed, the disorder or disease treated, the effect to be achieved, and the subject to be treated. Exemplary unit doses range from about 25-250, 250-500, 500-1,000, 1,000-2,500, 2,500-5,000, 5,000-25,000, or 5,000-50,000 pg; from about 50-500, 500-5,000, 5,000-25,000 or 25,000-50,000 ng; from about 50-500, 500-5,000, 5,000-25,000 or 25,000-50,000 □g; from about 25-250, 250-500, 500-1,000, 1,000-2,500, 2,500-5,000, 5,000-25,000, or 5,000-50,000 mg; and from about 1-5, 5-10, 10-25, 25-50, 50-100, 100-250, 250-500, 500-1,000, 1,000-2,500, or 2,500-5,000 grams.

As set forth herein, humanin and/or humanin analogs and compositions thereof may be contacted or provided in vitro, ex vivo or administered or delivered in vivo to a subject or patient in various doses and amounts, and frequencies. For example, a humanin and/or humanin analog or a composition thereof can be administered or delivered to provide the intended effect, as a single or as multiple dosages, for example, in an effective or sufficient amount.

Single or multiple (e.g., 2, 3, 4, 5, 6, 7, 8, 9, 10, or more times) administrations or doses can be administered on the same or consecutive days, alternating days or intermittently. For example, a humanin and/or humanin analog or a composition thereof can be administered one, two, three, four or more times daily, on alternating days, bi-weekly, weekly, monthly, bi-monthly, or annually. A humanin and/or humanin analog or composition thereof can be administered for any appropriate duration, for example, for period of 1 hour, or less, e.g., 30 minutes or less, 15 minutes or less, 5 minutes or less, or 1 minute, or less.

A humanin and/or humanin analog or composition thereof can be administered to a subject and methods and uses may be practiced prior to, substantially contemporaneously with, or within about 1-60 minutes, hours (e.g., within 1, 2, 3, 4, 5, 6, 8, 12, 24 hours), or days (1, 2, 3, 4, 5, 6, 7, 7-14, 14-21, 21-28, 28-45, 45-60, 60-90, etc.) of administration of a therapeutic agent, pathogenic agent and/or mechanical activity.

A humanin and/or humanin analog or composition thereof can be administered or delivered and methods and uses may be practiced via systemic, regional or local administration, by any route. For example, a humanin and/or humanin analog or composition thereof may be administered or delivered systemically, regionally or locally, via injection, infusion, orally (e.g., ingestion or inhalation), topically, intravenously, intra-arterially, intramuscularly, intraperitoneally, intradermally, subcutaneously, intracavity, intracranially, transdermally (topical), parenterally, e.g. transmucosally or intrarectally (enema) catheter, or optically. Humanin and/or humanin analog, compositions, methods and uses of the invention including pharmaceutical formulations may be administered via a (micro)encapsulated delivery system or packaged into an implant for administration.

Humanin and/or humanin analog or composition thereof can be incorporated into pharmaceutical compositions, e.g., a pharmaceutically acceptable carrier or excipient. Such pharmaceutical compositions are useful for, among other things, administration and delivery to a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in a formulation for administering or delivering a humanin and/or humanin analog or composition thereof to a subject.

Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Carriers which may be used include petroleum jelly, lanolin, polyethylene glycols, alcohols, transdermal enhancers, and combinations thereof.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Appropriate pharmaceutical compositions and delivery systems are known in the art (see, e.g., Remington: The Science and Practice of Pharmacy (2003) 20^{th} ed., Mack Publishing Co., Easton, Pa.; Remington's Pharmaceutical Sciences (1990) 18^{th} ed., Mack Publishing Co., Easton, Pa.; The Merck Index (1996) 12^{th} ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, Md.; and Poznansky et al., Drug Delivery Systems (1980), R. L. Juliano, ed., Oxford, N.Y., pp. 253-315).

The invention provides kits comprising humanin and/or humanin analogs, combination compositions and pharmaceutical formulations thereof, packaged into suitable packaging material. A kit optionally includes a label or packaging insert including a description of the components or instructions for use in vitro, in vivo, or ex vivo, of the components therein. Exemplary instructions include instructions for a method, treatment protocol or therapeutic regimen. The term “packaging material” refers to a physical structure housing the components of the kit. The packaging material can maintain the components sterilely, and can be made of material commonly used for such purposes (e.g., paper, corrugated fiber, glass, plastic, foil, ampules, vials, tubes, etc.).

Kits can include labels or inserts. Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards.

Labels or inserts can include identifying information of one or more components therein, dose amounts, clinical pharmacology of the active ingredient(s) including mechanism of action, pharmacokinetics (PK) and pharmacodynamics (PD). Labels or inserts can include information identifying manufacturer information, lot numbers, manufacturer location and date.

Labels or inserts can include information on a condition, disorder, disease or symptom for which a kit component may be used. Labels or inserts can include instructions for the clinician or for a subject for using one or more of the kit components in a method, treatment protocol or therapeutic regimen. Instructions can include dosage amounts, frequency or duration, and instructions for practicing any of the methods or uses, treatment protocols or therapeutic regimes set forth herein. Kits of the invention therefore can additionally include labels or instructions for practicing any of the methods and uses of the invention described herein.

Labels or inserts can include information on any benefit that a component may provide, such as a prophylactic or therapeutic benefit. Labels or inserts can include information on potential adverse side effects, such as warnings to the subject or clinician regarding situations where it would not be appropriate to use a particular composition. Adverse side effects could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Kits can additionally include other components. Each component of the kit can be enclosed within an individual container and all of the various containers can be within a single package. Invention kits can be designed for cold storage.

Representative non-limiting examples of HN and HN analogs/variants that are contemplated in the invention methods, uses and compositions include the following:

### Humanin (HN) Analogs and Variants (Tables 1-5)

TABLE 1
HN Variants with characteristics and cytoprotective action

| HN | | | |
|---|---|---|---|
| Mutant | Mutation | Characteristics | Cytoprotective Action |
| HN-F6A | Phe6 to Ala | Loss of IGFBP-3 | Similar/more effective |
| | | binding | than HN |
| HN-S7A | Ser7 to Ala | Loss of membrane | Not effective, prevents |
| or | Cys8 to Ala | receptor binding | HN self-dimerization |
| HN-C8A | | | |
| HN-C8P | Cys8 to Pro | Loss of BAX | Not effective, blocks |
| | | binding | intracellular HN action |
| HN-L12A | Leu12 to | Dimerizes with | HN antagonist, forms |
| | Ala | and inactivates | inactive dimer with HN |
| | | HN | |
| HN-S14G | Ser14 to | Same mechanisms | 10 to 1000 more potent |
| | Gly | of action as HN | than HN in some cells. |

TABLE 2
Additional HN Variants

| Name | Amino acid sequence | SEQ ID NO |
|---|---|---|
| Humanin (HN) | MAPRGFSCLLLLTSEIDLPVKRRA | SEQ ID NO: 1 |
| S14G-HN (HNG) | MAPRGFSCLLLLTGEIDLPVKRRA | SEQ ID NO: 2 |
| D-Ser14 HN | MAPRGFSCLLLLT(DS)EIDLPVKRRA | SEQ ID NO: 3 |
| AGA-HNG | MAPAGASCLLLLTGEIDLPVKRRA | SEQ ID NO: 4 |
| AGA-(D-Ser14)HN | MAPAGASCLLLLT(DS)EIDLPVKRRA | SEQ ID NO: 5 |
| AGA-(D- | PAGASCLLLLT(DS)EIDLP | SEQ ID NO: 6 |
| Ser14)HN17 | | |
| AGA-(C8R)HNG17 | PAGASRLLLLTGEIDLP | SEQ ID NO: 7 |
| EF-HN | EFLIVIKSMAPRGFSCLLLLTSEIDLPVKRRA | SEQ ID NO: 8 |
| EF-HNG | EFLIVIKSMAPRGFSCLLLLTGEIDLPVKRRA | SEQ ID NO: 9 |
| EF-AGA-HNG | EFLIVIKSMAPAGASCLLLLTGEIDLPVKRRA | SEQ ID NO: |
| | | 10 |
| Colivelin | SALLRSIPAPAGASRLLLLTGEIDLP | SEQ ID NO: |
| | | 11 |
| L9R-HN | MAPRGFSCRLLLTSEIDLPVKRRA | SEQ ID NO: |
| | | 12 |
| Humanin (7) | MTPRGFSCLLLPTSETDLPVKRRX | SEQ ID NO: |
| | | 13 |
| Humanin (5) | MAPRGFSCLLLSTSEIDLPVKRXX | SEQ ID NO: |
| | | 14 |
| Humanin (3/11) | MAPRGFSCLLLSTSEIDLPVKRRA | SEQ ID NO: |
| | | 15 |
| SHLP1 | MCHWAGGASNTGDARGDVFGKQAG | SEQ ID NO: |
| | | 16 |
| SHLP2 | MGVKFFTLSTRFFPSVQRAVPLWTNS | SEQ ID NO: |
| | | 17 |
| SHLP3 | MLGYNFSSFPCGTISIAPGFNFYRLYFIWVNGLAKVVW | SEQ ID NO: |
| | | 18 |
| SHLP4 | MLEVMFLVNRRGKICRVPFTFFNLSL | SEQ ID NO: |
| | | 19 |
| SHLP5 | MYCSEVGFCSEVAPTEIFNAGLVV | SEQ ID NO: |
| | | 20 |
| SHLP6 | MLDQDIPMVQPLLKVRLFND | SEQ ID NO: |
| | | 21 |
| HN-F6A | MAPRGASCLLLLTSEIDLPVKRRA | SEQ ID NO: |
| | | 22 |
| HNG-F6A | MAPRGASCLLLLTGEIDLPVKRRA | SEQ ID NO: |
| | | 23 |
| HN-S7A | MAPRGFACLLLLTSEIDLPVKRRA | SEQ ID NO: |
| | | 24 |
| HN-C8P | MAPRGFSPLLLLTSEIDLPVKRRA | SEQ ID NO: |
| | | 25 |

TABLE 3
Further HN Variants

| | | |
|---|---|---|
| P-S14 HN 4 | MAPRGFSCLLLLT(p-S)EIDLPVKRRA | SEQ ID NO: 26 |
| P-S7 HN 5 | MAPRGF(p-S)CLLLLTSEIDLPVKRRA | SEQ ID NO: 27 |
| P-S7/14 HN 6 | MAPRGF(p-S)CLLLLT(p-S)EIDLPVKRRA | SEQ ID NO: 28 |
| D-Ser14 HN 7 | MAPRGFSCLLLLT(D-S)EIDLPVKRRA | SEQ ID NO: 29 |
| D-Ser7 HN 8 | MAPRGF(D-Ser)CLLLLTSEIDLPVKRRA | SEQ ID NO: 30 |
| D-Ser7/14 HN 9 | MAPRGF(D-Ser)CLLLLT(D-Ser)EIDLPVKRRA | SEQ ID NO: 31 |
| AGA-(D-Ser14) HN 10 | MAPAGASCLLLLT(D-Ser)EIDLPVKRRA | SEQ ID NO: 32 |
| AGA-(D-Ser14) HN17 11 | PAGASCLLLLT(D-Ser)EIDLP | SEQ ID NO: 33 |
| EF-(S7A)HN 15 | EFLIVIKSMAPRGFACLLLLTSEIDLPVKRRA | SEQ ID NO: 34 |
| EF-HNG-KKK 16 | EFLIVIKSMAPRGFSCLLLLTGEIDLPVKKKK | SEQ ID NO: 35 |
| EF-HN 17 | EFLIVIKSMAPRGFSCLLLLTSEIDLPVKRRA | SEQ ID NO: 36 |
| EH-HNA 18 | EFLIVIKSMAPRGFSALLLLTSEIDLPVKRRA | SEQ ID NO: 37 |
| EF-HNG 19 | EFLIVIKSMAPRGFSCLLLLTGEIDLPVKRRA | SEQ ID NO: 38 |
| EF-AGA-HNG 22 | EFLIVIKSMAPAGASCLLLLTGEIDLPVKRRA | SEQ ID NO: 39 |

TABLE 4

| | | |
|---|---|---|
| HN 1 | MAPRGFSCLLLLTSEIDLPVKRRA | SEQ ID NO: 40 |
| HNG 2 | MAPRGFSCLLLLTGEIDLPVKRRA | SEQ ID NO: 41 |
| HNA 3 | MAPRGFSALLLLTSEIDLPVKRRA | SEQ ID NO: 42 |
| P-S14 HN 4 | MAPRGFSCLLLLT(p-S)EIDLPVKRRA | SEQ ID NO: 43 |
| P-S7 HN 5 | MAPRGF(p-S)CLLLLTSEIDLPVKRRA | SEQ ID NO: 44 |
| P-S7/14 HN 6 | MAPRGF(p-S)CLLLLT(p-S)EIDLPVKRRA | SEQ ID NO: 45 |
| D-Ser14 HN 7 | MAPRGFSCLLLLT(D-S)EIDLPVKRRA | SEQ ID NO: 46 |
| D-Ser7 HN 8 | MAPRGF(D-Ser)CLLLLTSEIDLPVKRRA | SEQ ID NO: 47 |
| D-Ser7/14 HN 9 | MAPRGF(D-Ser)CLLLLT(D-Ser)EIDPPVKRRA | SEQ ID NO: 48 |
| AGA-(D-Ser14) HN 10 | MAPAGASCLLLLT(D-Ser)EIDLPVKRRA | SEQ ID NO: 49 |
| AGA-(D-Ser14) HN17 11 | PAGASCLLLLT(D-Ser)EIDLP | SEQ ID NO: 50 |
| S7A HN 12 | MAPRGFACLLLLTSEIDLPVKRRA | SEQ ID NO: 51 |
| S7A HNG17 13 | PRGFACLLLLTSEIDLP | SEQ ID NO: 52 |
| HNG-KKK 14 | YMAPRGFSCLLLLTGEIDLPVKKKK | SEQ ID NO: 53 |
| EF-(S7A)HN 15 | EFLIVIKSMAPRGFACLLLLTSEIDLPVKRRA | SEQ ID NO: 54 |
| EF-HNG-KKK 16 | EFLIVIKSMAPRGFSCLLLLTGEIDLPVKKKK | SEQ ID NO: 55 |
| EF-HN 17 | EFLIVIKSMAPRGFSCLLLLTSEIDLPVKRRA | SEQ ID NO: 56 |
| EH-HNA 18 | EFLIVIKSMAPRGFSALLLLTSEIDLPVKRRA | SEQ ID NO: 57 |
| EF-HNG 19 | EFLIVIKSMAPRGFSCLLLLTGEIDLPVKRRA | SEQ ID NO: 58 |
| EFLIVIKS 20 | EFLIVIKS | SEQ ID NO: 59 |
| AGA-HNG 21 | MAPAGASCLLLLTGEIDLPVKRRA | SEQ ID NO: 60 |
| EF-AGA-HNG 22 | EFLIVIKSMAPAGASCLLLLTGEIDLPVKRRA | SEQ ID NO: 61 |
| HNG-17 23 | PRGFSCLLLLTGEIDLP | SEQ ID NO: 62 |

Table 5:

[symbol]

HNG: An HN derivative, which has a Gly substitution of Ser14 of HN.

HN derivatives can be selected from: Humanin with S14P, P-S7 HN, P-S7/14 HN, (D-Ser14)HN, (D-Ser7)HN, (D-Ser7/14)HN, AGA-(D-Ser14)HN, AGA-(D-Ser14)HN17, EFLIVIKS-HNG, EFLIVIKS-HNA, EFLIVIKS-HN, EFLIVIKS-HNG-KKK, EFLIVIKS-(S7A)HN, and EFLIVIKS-AGA-HNG, and chimeric combinations thereof The “S14P” means that the S (serine) at location 14 in the wild-type HN has been replaced with P (proline). The same convention applies for other substitutions (e.g., S7A). “D-Ser7” means that the Serine at location 7 has been switched (racemized) from a normal L-isomer to the D-isomer. “AGA-HN” is a shorthand name of the HN derivative in which the Arg4 and Phe6 amino acids are substituted with Alanine to form R4A/F6A-HN (this is named for the AGA triplet at locations 4, 5, and 6 in the HN derivative. “HN17” is a truncated form of HN that includes 17 amino acids from Pro3 to Pro19.

A polypeptide having an amino acid sequence of: Pro-Xn1-(Cys/bXaa)-(Leu/Arg)-Xn2-Leu-Thr-(Gly/Ser)-Xn3-Pro (I) wherein “Cys/bXaa” indicates Cys or a basic amino acid; “(Leu/Arg)” indicates Leu or Arg; “(Gly/Ser)” indicates Gly or Ser; and Xn1, Xn2, and Xn3 independently indicate arbitrary amino acid sequences not more than 10 residues in length, respectively;

A polypeptide having an amino acid sequence of: Pro-Xn1-(Cys/bXaa)-(Leu/Arg)-Xn2-Leu-Thr-(Gly/Ser)-Xn3-Pro (1).

Herein, “Cys/bXaa” indicates Cys or a basic amino acid; “(Leu/Arg)” indicates Leu or Arg; “(Gly/Ser)” indicates Gly or Ser; and Xn1, Xn2, and Xn3 independently indicate arbitrary amino acids not more than 10 residues, respectively.

A polypeptide that has the amino acid sequence of: Pro-(Xaa)1-10-(Cys/bXaa)-(Leu/Arg)-(Xaa)1-10-Leu-Thr-(Gly/Ser)-(Xaa)1-10Pro (wherein Xaa indicates an arbitrary amino acid; “(Xaa)m-n” indicates m to n residues of arbitrary amino acids; “bXaa” indicates a basic amino acid; “Cys/bXaa” indicates Cys or a basic amino acid; “(Leu/Arg)” indicates Leu or Arg; and “(Gly/Ser)” indicates Gly or Ser).

Basic amino acids refer to amino acids in which its R group (side chain) is positively charged at pH7.0. Examples of natural basic amino acids include Arg, Lys, and His.

The amino acid sequences of a polypeptide that has Arg, Lys, or His as the basic amino acids can be represented, for example, as: Pro-Xn1-(Cys/Arg/Lys/His)-(Leu/Arg)-Xn2-Leu-Thr-(Gly/Ser)-Xn3-Pro (wherein “(Cys/Arg/Lys/His)” indicates Cys, Arg, Lys, or His; “(Leu/Arg)” indicates Leu or Arg; “(Gly/Ser)” indicates Gly or Ser; and Xn1, Xn2, and Xn3 independently indicate arbitrary amino acids not more than 10 residues, respectively). Herein, Arg and Lys are particularly preferable as the basic amino acid at this position.

Xn1, Xn2, and Xn3 are independently arbitrary amino acids of 2 to 6, 0 to 4, and 2 to 6 residues, respectively (that is, Xn1=(Xaa)2-6, Xn2=(Xaa)0-4, and Xn3=(Xaa)2-6); more preferably 3 to 5, 1 to 3, and 3 to 5 residues, respectively (that is, Xn1=(Xaa)3-5, Xn2=(Xaa)1-3, and Xn3=(Xaa)3-5); and most preferably 4, 2, and 4 residues, respectively (that is, Xn1=(Xaa)4, Xn2=(Xaa)2, and Xn3=(Xaa)). Added amino acids of about 6 residues sometimes forms an α-helix and behaves like a single amino acid residue. A polypeptide of the present invention may be a polypeptide wherein arbitrary amino acids with no more than 6 residues are added to all or any one of Xn1, Xn2, and Xn3 consisting of arbitrary amino acids of 4 residues, 2 residues, and 4 residues, respectively.

A sequence of Xn1 includes, for example, sequences consisting of (Arg/Ala)-(Gly/Ala)-(Phe/Ala)-(Ser/Ala), and sequences with conservative substitution thereof. Herein, for example, “Arg/Ala” indicates Arg or Ala (“/” indicates that it is either one of the residues; the same is indicated throughout the description herein). Examples of such sequences include Arg-Gly-Phe-Ser (SEQ ID NO: 63), Ala-Gly-Phe-Ser (SEQ ID NO: 64), Arg-Ala-Phe-Ser (SEQ ID NO: 65), Arg-Gly-Ala-Ser (SEQ ID NO: 66), Arg-Gly-Phe-Ala (SEQ ID NO: 67), and so on. Other examples include Arg-Gly-Ala-Ala (SEQ ID NO: 68), Arg-Ala-Phe-Ala (SEQ ID NO: 69), Arg-Ala-Ala-Ser (SEQ ID NO: 70), Arg-Ala-Ala-Ala (SEQ ID NO: 71), Ala-Gly-Phe-Ala (SEQ ID NO: 72), Ala-Gly-Ala-Ser (SEQ ID NO: 73), Ala-Gly-Ala-Ala (SEQ ID NO: 74), Ala-Ala-Phe-Ser (SEQ ID NO: 75), Ala-Ala-Phe-Ala (SEQ ID NO: 76), Ala-Ala-Ala-Ser (SEQ ID NO: 77), Ala-Ala-Ala-Ala (SEQ ID NO: 78), and such.

Conservative substitution can be exemplified by substitution within a group of amino acids, corresponding to conservative substitution, which will be described later. On the other hand, the sequence of Xn2 preferably includes, for example, sequences consisting of (Leu/Ala)-(Leu/Ala), and sequences with conservative substitution thereof. Such sequences include Leu-Leu, Ala-Leu, Leu-Ala, and such. Ala-Ala can be also exemplified as such sequences. Furthermore, the sequence of Xn3 preferably includes, for example, sequences consisting of (Glu/Ala)-(Ile/Ala)-(Asp/Ala)-(Leu/Ala), and sequences with conservative substitution thereof. Such examples include Glu-Ile-Asp-Leu (SEQ ID NO: 79), Ala-Ile-Asp-Leu (SEQ ID NO: 80), Glu-Ala-Asp-Leu (SEQ ID NO: 81), Glu-Ile-Ala-Leu (SEQ ID NO: 82), Glu-Ile-Asp-Ala (SEQ ID NO: 83), and so on. Other examples are Glu-Ile-Ala-Ala (SEQ ID NO: 84), Glu-Ala-Asp-Ala (SEQ ID NO: 85), Glu-Ala-Ala-Leu (SEQ ID NO: 86), Glu-Ala-Ala-Ala (SEQ ID NO: 87), Ala-Ile-Asp-Ala (SEQ ID NO: 88), Ala-Ile-Ala-Leu (SEQ ID NO: 89), Ala-Ile-Ala-Ala (SEQ ID NO: 90), Ala-Ala-Asp-Leu (SEQ ID NO: 91), Ala-Ala-Asp-Ala (SEQ ID NO: 92), Ala-Ala-Ala-Leu (SEQ ID NO: 93), Ala-Ala-Ala-Ala (SEQ ID NO: 94), and so on. The sequences of Xn1, Xn2, and Xn3 may be selected from arbitrary combinations.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All applications, publications, patents and other references, GenBank citations and ATCC citations cited herein are incorporated by reference in their entirety. In case of conflict, the specification, including definitions, will control.

All of the features disclosed herein may be combined in any combination. Each feature disclosed in the specification may be replaced by an alternative feature serving a same, equivalent, or similar purpose. Thus, unless expressly stated otherwise, disclosed features (e.g., humanin and humanin analogs) are an example of a genus of equivalent or similar features.

As used herein, the singular forms “a,” “and,” and “the” include plural referents unless the context clearly indicates otherwise. Thus, for example, reference to “a humanin” includes a plurality of humanin molecules.

As used herein, all numerical values or numerical ranges include integers within such ranges and fractions of the values or the integers within ranges unless the context clearly indicates otherwise. Thus, to illustrate, reference to at least 100%, includes 101%, 102%, 103%, 104%, 105%, etc., as well as 101.1%, 101.2%, 101.3%, 101.4%, 101.5%, etc.

Reference to an integer with more (greater) or less than includes any number greater or less than the reference number, respectively. Thus, for example, a reference to less than 1,000, includes 999, 998, 997, etc. all the way down to the number one (1), and fractions thereof (e.g., 0.5, 0.1, 0.05, 0.01, etc.; and less than 100, includes 99, 98, 97, etc. all the way down to the number one (1), and fractions thereof (e.g., 0.5, 0.1, 0.05, 0.01, etc.).

As used herein, all numerical values or ranges include fractions of the values and integers within such ranges and fractions of the integers within such ranges unless the context clearly indicates otherwise. Thus, to illustrate, reference to a numerical range, such as a percentage range, such as 50-100 includes 51, 52, 53, 54, 55, 56, 57, 58, 59, 60, as well as 51.1, 51.2, 51.3, 51.4, 51.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges of 11-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed.

### EXAMPLES

### 1. Materials and Methods

### Animals

Adult male C57BL/6J (25 to 30g) mice were obtained from Jackson Laboratory (Bar Harbor, Me.) and housed at the accredited animal facilities at Los Angeles Biomedical Research Institute. The mice had unlimited access to food and water and were provided housing at normal light-dark cycles (12 h each) at a constant temperature of 22° C. Animal handling, experimentation, and killing of the animals were in accordance with the recommendation of the American Veterinary Medical Association and were approved by the Animal Care and Use Review Committee of Los Angeles Biomedical Research Institute at Harbor-UCLA Medical Center.

### Materials

HN and TING were synthesized by CPC Scientific (Sunnyvale, Calif.), and DOX (doxorubicin hydrochloride) and CP (cyclophosphamide monohydrate) were obtained from Sigma Aldrich (St. Louis, Mo.). Both HNG and IP were administered as intraperitoneal injections (IP).

### Blood Collection and Tissue Preparation

Mice were injected with heparin (1300 IU/kg BW, ip) 15 min before sacrifice by sodium pentobarbital (200 mg/kg BW IP). Body weight (BW) was recorded at autopsy. Blood samples were collected from the right ventricle of each mouse immediately after death and used for complete blood count using an automated cell counter (VetScanHM2, ABAXIS, Union City, Calif.). Plasma was separated and stored at −20° C. for subsequent HNG, IGF-1 and IGFBP-1 measurements by specific and sensitive ELISA assays as previously described (Chin et al., Endocrinology. 2013; 154:3739-44; Hwang et al, Growth Horm IGF Res 2008; 18: 65-74). The humanin ELISA was used to measure plasma HNG levels because the antibody used in the assay cross-reacted completely with HNG.

### Statistical Analyses

Statistical analyses were carried out using the SigmaStat 12.0 Program (Systat Software Inc, San Jose, Calif.). Results were tested for statistical significance using one-way ANOVA with post-hoc Tukey test or Student t test. Differences were considered significant if P value was less than 0.05.

### 2. HN WBC Protection Studies

### 2.1 Adult Normal Mice

The effect of a potent HN analog HNG (HN-S14G) (5mg/KG/day, IP) with and without concomitant cyclophosphamide (CP, 200 mg IP) treatment was administered to 4 adult healthy mice in each group. Another four mice served as controls. Two days after treatment, the animals were killed and their peripheral blood cells were collected and the blood cell counts were assessed by a blood cell counter.

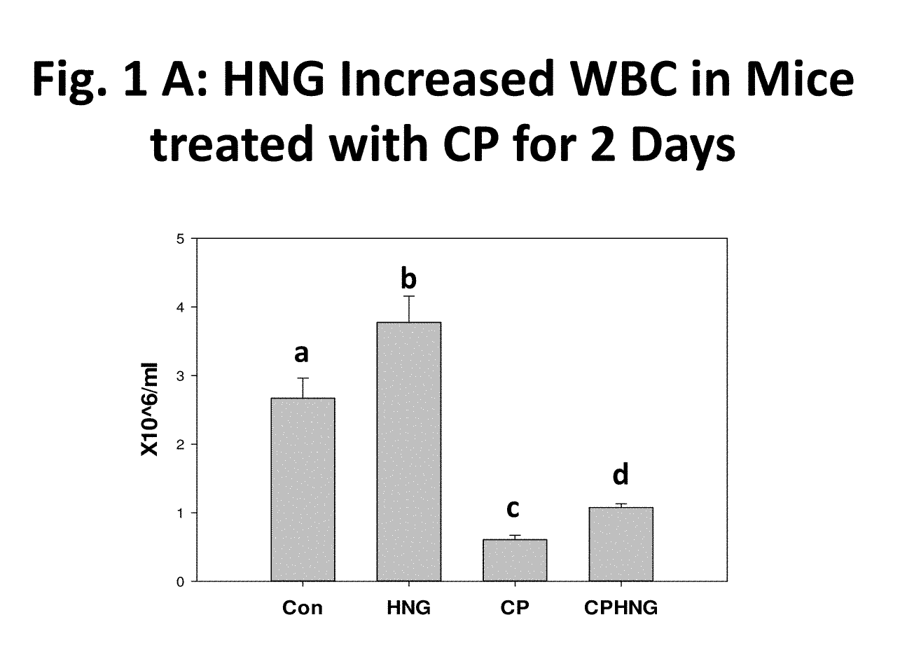

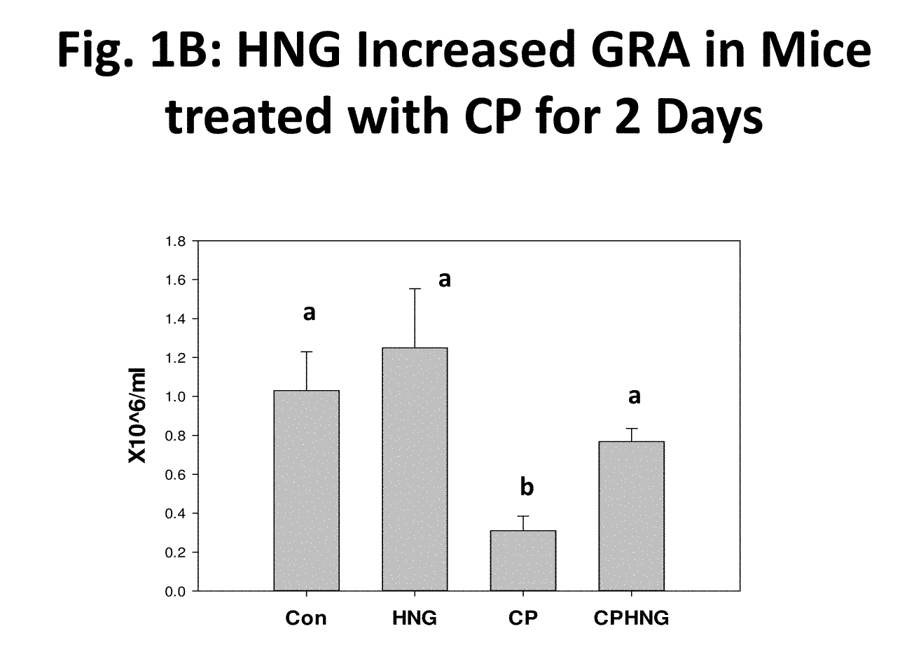

As shown in FIG. 1A, the total number of white blood cells (WBC) were increased slightly but significantly by HN treatment alone. As anticipated, CP treatment drastically reduced WBC about ⅕ of the baseline counts (similar findings in man administered CP). Treatment with TING reduced the CP induced suppression of peripheral WBC. Importantly, the granulocyte count in the blood was reduced to about 25% of baseline value two days after CP treatment. Concomitant treatment with HNG prevented the marked decrease in granulocyte counts induced by CP (FIG. 1B). The effects of three days pre-treatment with HNG before CP to prevent the CP suppression of peripheral WBC count was determined.

### 2.2 Melanoma Bearing Mice

The effect of a potent HN analog HNG (HN-S14G) with or without cyclophosphamide (CP) in a mouse melanoma with lung metastasis model was studied. Five mice were used as control (Con) and 40 mice were inoculated intravenously with B16 murine melanoma cells. Of these mice five received no further treatment (NT). A week later the remaining 35 mice (n=5 per group) were treated for additional 2 weeks with:
1) HNG 0.05 mg/KG body weight (BW) daily IP injection for 2 weeks;
2) HNG 0.5 mg mg/Kg BW daily IP injection for 2 weeks;
3) HNG 5 mg/Kg BW daily IP injection for 2 weeks;
4) a single CP (200 mg IP single dose) injection+HNG 0.05 mg/KG body weight (BW) daily IP injection for 2 weeks;
5) a single CP (200 mg IP single dose) injection+HNG 0.5 mg mg/Kg BW daily IP injection for 2 weeks;
6) a single CP (200 mg IP single dose) injection HNG 0.5 mg mg/Kg BW daily IP injection for 2 weeks;
7) a single CP (200 mg IP single dose) injection+HNG 5 mg mg/Kg BW daily IP injection.

All mice were sacrificed three weeks after tumor inoculation and two weeks after CP/HNG treatment. Blood was obtained for the measurement of white blood cell (WBC) count by a Coulter Counter.

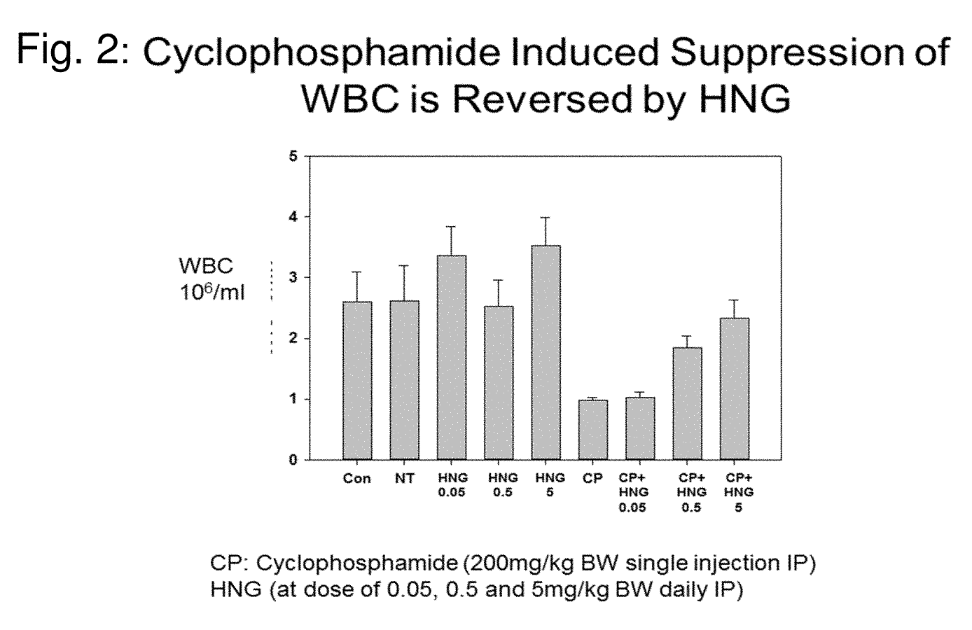

As shown in FIG. 2, HNG up to 5 mg/Kg BW IP per day for two weeks had no effect on white blood cell count (WBC). CP at the dose used significantly decreased WBC count to about ⅓ of control values. Concomitant administration of CP and HNG showed that at lower dose, HNG 0.05 mg/KG BW daily for two weeks had no detectable effect on the WBC suppression by CP. However, HNG at a dose of 0.5 mg/Kg/BW/day and 5 mg/KG/BW/day for two weeks reversed the WBC suppression by CP.

In addition to total WBC analysis, the effects of HNG on peripheral blood leucocytes, plasma IGF-1 and IGFBP-1 levels were analyzed confirming its additive or synergistic effects on CP-induced suppression of lung melanoma metastases in tumor-bearing mice

In this study the tumor loading dose was 1×10⁵ B16 cells per mouse to examine the effects of HNG on peripheral blood leucocytes, plasma IGF-1 and IGFBP-1 levels while confirming its additive or synergistic effect on CP-induced suppression of lung melanoma metastases. Twenty-five young adult (12-week-old) male mice (C57BL/6J) were used for this experiment in which 5 mice were used as control and 20 mice of each were intravenously inoculated via the tail vein with 1×10⁵ murine B16 melanoma cells. 20 tumor-bearing mice were randomly divided into 4 groups with 5 mice in each group received 1) no treatment; 2) HNG alone; 3) CP alone; and 4) a combination treatment with the doses and duration described above.

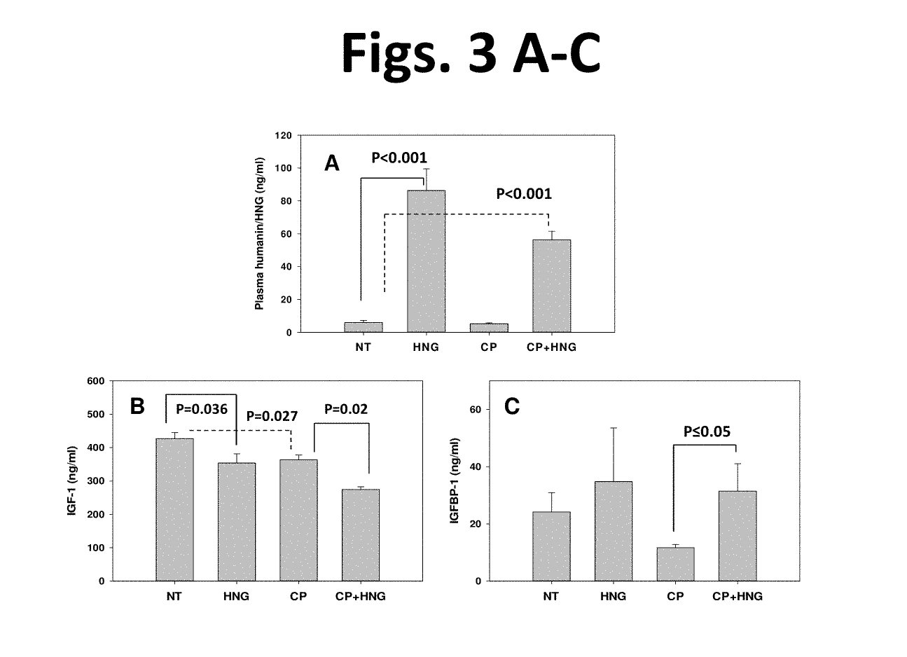

Administration of HNG increased plasma HNG levels (P<0.001) in both groups treated with exogenous HNG (HNG and HNG+CP groups) as compared to non-treated tumor-bearing (NT) and CP alone group (FIG. 3A). HNG (p=0.036) or CP (p=0.027) treatment alone significantly suppressed plasma IGF-1 levels. Addition of HNG to CP further suppressed IGF-1 (p=0.02) levels compared to CP alone (FIG. 3B). Co-treatment of HNG with CP increases plasma IGFBP-1 levels when compared to CP alone (p<0.05) (FIG. 3C). Data is summarized in Table 6.

TABLE 6
Comparison of plasma HNG, IGF-1 and IGFBP-1
levels among groups in healthy mice

| Names | Control | HNG | CP | CP + HNG |
|---|---|---|---|---|
| Number of Mice | 5 | 5 | 10 | 10 |
| HNG (ng/ml) | 1.3 ± 0.1^{a} | 80.8 ± 7.8^{b} | 1.7 ± 0.1^{a} | 64.7 ± 1.8^{b} |
| IGF-1 (ng/ml) | 413.7 ± 44.9^{a} | 347.2 ± 20.1^{b} | 182.4 ± 10.5^{c} | 148.8 ± 8.1^{d} |
| IGFBP-1 (ng/ml) | 27.1 ± 10.1^{a} | 29.7 ± 5.9^{a} | 7.4 ± 1.3^{b} | 18.5 ± 2.5^{a} |

Values are mean ± SEM. Groups with different superscripts are significantly different (P < 0.05).

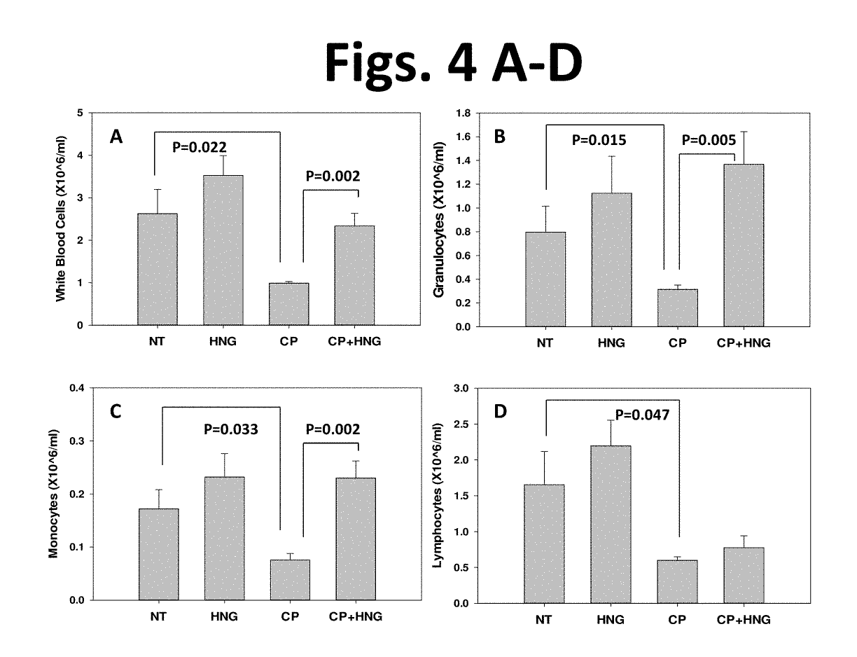

CP treatment significantly suppressed the number of white blood cells (FIG. 4A, p=0.022), granulocytes (FIG. 4B, p=0.015), monocytes (FIG. 4C, p=0.033) and lymphocytes (FIG. 4D, p=0.047) compared to non-treated tumor-bearing mice. Importantly, addition of HNG to CP significantly increased white blood cells (FIG. 4A, p=0.002), granulocytes (FIG. 4B, p=0.005), and monocytes (FIG. 4C, p=0.002) compared to CP treatment alone, restoring these cell counts to the levels observed in non-treated tumor-bearing mice. HNG+CP did not significantly rescue the decrease in lymphocytes induced by a single dose of CP (FIG. 4D). There were no significant decreases in red blood cell or platelet counts in the groups treated with CP and HNG+CP groups. Treatment with TING or CP significantly decreased (p<0.001) the number of metastatic tumors in the lungs as compared to non-treated tumor-bearing mice, addition of HNG to CP treatment further decreased number of tumors (p=0.013) compared to CP treatment alone. Data is summarized in Table 7.

TABLE 7
Comparison of peripheral leucocytes among
different treatment groups of healthy mice

| Names | Control | HNG | CP | CP + HNG |
|---|---|---|---|---|
| Number of Mice | 5 | 5 | 10 | 10 |
| Total WBC (10⁶/ml) | 2.4 ± 0.3^{a} | 2.3 ± 0.6^{a} | 0.3 ± 0.02^{b} | 0.6 ± 0.04^{c} |
| Granulocytes (10⁶/ml) | 0.2 ± 0.1^{ab} | 0.5 ± 0.2^{ad} | 0.2 ± 0.02^{b} | 0.4 ± 0.03^{cd} |
| Monocytes (10⁶/ml) | 0.1 ± 0.02^{a} | 0.1 ± 0.03^{a} | 0.01 ± 0.01^{b} | 0.05 ± 0.01^{c} |
| Lymphocytes (10⁶/ml) | 2.1 ± 0.4^{a} | 1.7 ± 0.4^{a} | 0.07 ± 0.01^{b} | 0.1 ± 0.01^{c} |

Values are mean ± SEM. Groups with different superscripts are significantly different (P < 0.05).

Accordingly, the data show that HNG suppresses plasma IGF-1 and increases IGFBP-1 levels, and significantly rescues the CP-induced suppression of peripheral leucocytes (white blood cells, granulocytes and monocytes) while enhancing CP-induced tumor suppression.

### REFERENCES

Bachar A R, Scheffer L, Schroeder A S, Nakamura H K, Cobb L J, Oh Y K, Lerman L O, Pagano R E, Cohen P & Lerman A. (2010) Humanin is expressed in human vascular walls and has a cytoprotective effect against oxidized LDL-induced oxidative stress. Cardiovasc. Res. 88, 360-366.
Chin Y P, Keni J, Wan J, Mehta H, Anene F, Jia Y, Lue Y H, Swerdloff R, Cobb L J, Wang C, Cohen P. Pharmacokinetics and tissue distribution of humanin and its analogues in male rodents. Endocrinology. 2013; 154:3739-44.
Choy J T & Brannigan R E. (2013) The determination of reproductive safety in men during and after cancer treatment. Fertil. Steril. 100, 1187-1191.
Collisson E A, De A, Suzuki H, Gambhir S S & Kolodney M S. (2003) Treatment of metastatic melanoma with an orally available inhibitor of the Ras-Raf-MAPK cascade. Cancer Res. 63, 5669-5673.
Colon E, Strand M L, Carlsson-Skwirut C, Wahlgren A, Svechnikov K V, Cohen P & Soder O. (2006) Anti-apoptotic factor humanin is expressed in the testis and prevents cell-death in leydig cells during the first wave of spermatogenesis. J. Cell. Physiol. 208, 373-385.
Craft N, Bruhn K W, Nguyen B D, Prins R, Liau L M, Collisson E A, De A, Kolodney M S, Gambhir S S & Miller J F. (2005) Bioluminescent imaging of melanoma in live mice. J. Invest. Dermatol. 125, 159-165.
Delbes G, Vaisheva F, Luu T, Marcon L, Hales B F & Robaire B. (2010) Reversibility of the effects of the chemotherapeutic regimen for non-Hodgkin lymphoma, cyclophosphamide, doxorubicin, vincristine, and prednisone, on the male rat reproductive system and progeny outcome. Reprod. Toxicol. 29, 332-338.
Dohle G R. (2010) Male infertility in cancer patients: Review of the literature. Int. J. Urol. 17, 327-331.
Drumond A L, Weng C C, Wang G, Chiarini-Garcia H, Eras-Garcia L & Meistrich M L. (2011) Effects of multiple doses of cyclophosphamide on mouse testes: accessing the germ cells lost, and the functional damage of stem cells. Reprod. Toxicol. 32, 395-406.
Erkkila K, Henriksen K, Hirvonen V, Rannikko S, Salo J, Parvinen M & Dunkel L. (1997) Testosterone regulates apoptosis in adult human seminiferous tubules in vitro. J. Clin. Endocrinol. Metab. 82, 2314-2321.
Hartmann T B, Mattern E, Wiedemann N, van Doom R, Willemze R, Niikura T, Hildenbrand R, Schadendorf D & Eichmuller S B. (2008) Identification of selectively expressed genes and antigens in CTCL. Exp. Dermatol. 17, 324-334.
Hashimoto Y, Niikura T, Ito Y, Sudo H, Hata M, Arakawa E, Abe Y, Kita Y & Nishimoto I. (2001) Detailed characterization of neuroprotection by a rescue factor humanin against various Alzheimer's disease-relevant insults. J. Neurosci. 21, 9235-9245.
Hashimoto Y, Niikura T, Tajima H, Yasukawa T, Sudo H, Ito Y, Kita Y, Kawasumi M, Kouyama K, Doyu M, Sobue G, Koide T, Tsuji S, Lang J, Kurokawa K & Nishimoto I. (2001) A rescue factor abolishing neuronal cell death by a wide spectrum of familial Alzheimer's disease genes and Abeta. Proc. Natl. Acad. Sci. U.S.A. 98, 6336-6341.
Hoang P T, Park P, Cobb L J, Paharkova-Vatchkova V, Hakimi M, Cohen P & Lee K W. (2010) The neurosurvival factor Humanin inhibits beta-cell apoptosis via signal transducer and activator of transcription 3 activation and delays and ameliorates diabetes in nonobese diabetic mice. Metabolism. 59, 343-349.
Hwang D L, Lee P D, Cohen P. Quantitative ontogeny of murine insulin-like growth factor (IGF)-I, IGF-binding protein-3 and the IGF-related acid-labile subunit. Growth Horm IGF Res 2008; 18: 65-74.
Jia Y, Lue Y H, Swerdloff R, Lee K W, Cobb L J, Cohen P & Wang C. (2013) The cytoprotective peptide humanin is induced and neutralizes Bax after pro-apoptotic stress in the rat testis. Andrology 1, 651-659.
Loren A W, Mangu P B, Beck L N, Brennan L, Magdalinski A J, Partridge A H, Quinn G, Wallace W H & Oktay K. (2013) Fertility preservation for patients with cancer: American Society of Clinical Oncology clinical practice guideline update. J. Clin. Oncol. 31, 2500-2510.
Lue Y, Swerdloff R, Liu Q, Mehta H, Hikim A S, Lee K W, Jia Y, Hwang D, Cobb L J, Cohen P & Wang C. (2010) Opposing roles of insulin-like growth factor binding protein 3 and humanin in the regulation of testicular germ cell apoptosis. Endocrinology 151, 350-357.
Lue Y, Wang C, Cui Y, Wang X, Sha J, Zhou Z, Xu J, Hikim A P & Swerdloff R S. (2009) Levonorgestrel enhances spermatogenesis suppression by testosterone with greater alteration in testicular gene expression in men. Biol. Reprod. 80, 484-492.
Lue Y H, Wang C, Liu P Y, Erkilla K & Swerdloff R S. (2010) Insights into the pathogenesis of XXY phenotype from comparison of the clinical syndrome with an experimental XXY mouse model. Pediatr Endocrinol Rev 8 Suppl 1, 140-144.
Marcon L, Zhang X, Hales B F, Robaire B & Nagano M C. (2011) Effects of Chemotherapeutic Agents for Testicular Cancer on Rat Spermatogonial Stem/Progenitor Cells. J. Androl.
Maximov V, Martynenko A, Hunsmann G & Tarantul V. (2002) Mitochondrial 16S rRNA gene encodes a functional peptide, a potential drug for Alzheimer's disease and target for cancer therapy. Med. Hypotheses 59, 670-673.
Meistrich M L. (2013) Effects of chemotherapy and radiotherapy on spermatogenesis in humans. Fertil. Steril. 100, 1180-1186.
Meistrich M L, Finch M, da Cunha M F, Hacker U & Au W W. (1982) Damaging effects of fourteen chemotherapeutic drugs on mouse testis cells. Cancer Res. 42, 122-131.
Moretti E, Giannerini V, Rossini L, Matsuoka M, Trabalzini L & Collodel G. (2010) Immunolocalization of humanin in human sperm and testis. Fertil. Steril. 94, 2888-2890.
Mottaghi-Dastjerdi N, Soltany-Rezaee-Rad M, Sepehrizadeh Z, Roshandel G, Ebrahimifard F & Setayesh N. (2014) Genome expression analysis by suppression subtractive hybridization identified overexpression of Humanin, a target gene in gastric cancer chemoresistance. Daru: journal of Faculty of Pharmacy, Tehran University of Medical Sciences 22, 14.
Muzumdar R H, Huffman D M, Calvert J W, Jha S, Weinberg Y, Cui L, Nemkal A, Atzmon G, Klein L, Gundewar S, Ji S Y, Lavu M, Predmore B L & Lefer D J. (2010) Acute humanin therapy attenuates myocardial ischemia and reperfusion injury in mice. Arterioscler. Thromb. Vac. Biol. 30, 1940-1948.
Nangia A K, Krieg S A & Kim S S. (2013) Clinical guidelines for sperm cryopreservation in cancer patients. Fertil. Steril. 100, 1203-1209.
Sloderbach A, Gorska A, Sikorska M, Misiura K & Hladon B. (2013) Classical oxazaphosphorines—metabolism and therapeutic properties—new implications. Postepy higieny i medycyny doswiadczalnej (Online) 67, 1235-1253.
Tarantul V Z & Hunsmann G. (2001) Mitochondrial polypeptides of the oxidative phosphorylation pathway as potential new targets for anti-cancer therapy. Med. Hypotheses 56, 386-387.
Toppari J & Parvinen M. (1985) In vitro differentiation of rat seminiferous tubular segments from defined stages of the epithelial cycle morphologic and immunolocalization analysis. J. Androl. 6, 334-343.
Trost L W & Brannigan R E. (2012) Oncofertility and the male cancer patient. Current treatment options in oncology 13, 146-160.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation, and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed can be resorted to by those skilled in the art, and such modifications and variations are considered within the scope of this technology.

Certain embodiments of the technology are set forth in the claim(s) that follow(s).

What is claimed is:

1. A method of protecting, reducing, decreasing, or inhibiting white blood cells (WBC) in a subject from an autoimmune, anti-cancer or anti-tumor therapeutic agent suppression or death, wherein white blood cell suppression or death is induced, promoted, increased, or stimulated by an alkylating agent, comprising administering to a subject prior to, during or after treatment with an autoimmune, anti-cancer or anti-tumor therapeutic agent an amount of humanin or a humanin analog sufficient to protect, reduce, decrease, or inhibit white blood cells in the subject from suppression or death induced, promoted, increased, or stimulated by the autoimmune, anti-cancer or anti-tumor therapeutic agent.

2. A method of promoting or increasing maturation, proliferation or survival of white blood cells (WBC) in a subject, wherein white blood cell maturation, proliferation or survival is reduced, decreased, or inhibited by an autoimmune, anti-cancer or anti-tumor therapeutic agent, comprising administering to a subject prior to, during or after treatment with the autoimmune, anti-cancer or anti-tumor therapeutic agent an amount of humanin or a humanin analog sufficient to promote or increase maturation, proliferation or survival of white blood cells in the subject.

3. A method of reducing, decreasing, or inhibiting damage to white blood cells (WBC) in a subject by an autoimmune, anti-cancer or anti-tumor therapeutic agent, wherein white blood cells are damaged by an autoimmune, anti-cancer or anti-tumor therapeutic agent, comprising administering to a subject prior to, during or after treatment with the autoimmune, anti-cancer or anti-tumor therapeutic agent an amount of humanin or a humanin analog sufficient to reduce, decrease, or inhibit damage of white blood cells in the subject.

4. A method of reducing, decreasing, or inhibiting infection of a subject treated with an autoimmune, anti-cancer or anti-tumor therapeutic agent, comprising administering to a subject prior to, during or after treatment with the autoimmune, anti-cancer or anti-tumor therapeutic agent an amount of humanin or a humanin analog sufficient to reduce, decrease, or inhibit infection of the subject.

5. Use of humanin or a humanin analog in the manufacture of a medicament: 1) for protecting white blood cells (WBC) from autoimmune, anti-cancer or anti-tumor therapeutic agent suppression or death; 2) for promoting or increasing maturation, proliferation or survival of white blood cells (WBC), where white blood cell maturation, proliferation or survival is reduced, decreased, or inhibited by an autoimmune, anti-cancer or anti-tumor therapeutic agent; 3) to reduce, decrease, or inhibit damage of white blood cells caused by an autoimmune, anti-cancer or anti-tumor therapeutic agent; or 4) to reduce, decrease, or inhibit infection of a subject treated with an autoimmune, anti-cancer or anti-tumor therapeutic agent.

6. The method or use of claim 1 or 5, wherein the protecting, reducing, decreasing, or inhibiting from suppression or death provided is relatively greater for white blood cells (WBC) of myeloid lineage than of lymphoid lineage.

7. The method or use of claim 1 or 5, wherein the protecting, reducing, decreasing, or inhibiting from suppression or death provided is relatively greater for granulocytes and/or monocytes than for lymphocytes.

8. The method or use of any of claims 1-5, wherein the subject has a hyperproliferative or autoimmune disease or disorder.

9. The method or use of any of claims 1-5, wherein the subject has a metastatic or non-metastatic neoplasia, tumor, cancer or malignancy.

10. The method or use of any of claims 1-5, wherein the autoimmune, anti-cancer or anti-tumor therapeutic agent comprises an alkylating agent, an anthracycline, an anti-metabolite, plant extract, plant alkaloid, nitrosourea, hormone, nucleoside or nucleotide analog.

11. The method or use of any of claims 1-5, wherein the autoimmune, anti-cancer or anti-tumor therapeutic agent comprises a DNA intercalating agent or an agent that attaches or bonds to DNA.

12. The method or use of any of claims 1-5, wherein the autoimmune, anti-cancer or anti-tumor therapeutic agent comprises cyclophosphamide, acrolein, phosphoramide, doxorubicin, azathioprine, cyclosporin A, prednisolone, melphalan, chlorambucil, mechlorethamine, busulphan, methotrexate, 6-mercaptopurine, thioguanine, 5-fluorouracil, cytosine arabinoside, 5-azacytidine (5-AZC) and 5-azacytidine related compounds, bleomycin, actinomycin D, mithramycin, mitomycin C, carmustine, lomustine, semustine, streptozotocin, hydroxyurea, cisplatin, carboplatin, oxiplatin, mitotane, procarbazine, dacarbazine, a taxane, vinblastine, vincristine, dibromomannitol, gemcitabine, or pemetrexed.

13. The method or use of any of claims 1-5, wherein the humanin or a humanin analog does not substantially reduce, decrease, suppress or inhibit efficacy or activity of the autoimmune, anti-cancer or anti-tumor therapeutic agent.

14. The method or use of claim 13, wherein the efficacy or activity of the autoimmune, anti-cancer or anti-tumor therapeutic agent comprises partial or complete destruction of a hyperproliferating cell, or a neoplastic, tumor, cancer or malignant cell mass, volume, size or numbers of cells; stimulating, inducing or increasing hyperproliferating cell or neoplastic, tumor, cancer or malignant cell necrosis, lysis or apoptosis; reduces hyperproliferating cell or neoplasia, tumor, cancer or malignancy volume size or cell mass; inhibits or prevents progression or an increase in hyperproliferating cell or neoplasia, tumor, cancer or malignancy volume, mass, size or cell numbers, reduces neoplasia, tumor, cancer or malignancy metastasis volume, size or cell mass; or prolongs lifespan.

15. The method or use of any of claims 1-5, wherein the humanin comprises the sequence: MAPRGFSCLLLLTSEIDLPVKRRA.

16. The method of any of claims 1-5, wherein the humanin analog comprises the sequence: MAPRGFSCLLLLTGEIDLPVKRRA (HN-S14G), or any sequence set forth in Tables 1-5.

17. The method or use of claim 9, wherein the neoplasia, tumor, cancer or malignancy is metastatic, non-metastatic or benign.

18. The method or use of claim 9, wherein the neoplasia, tumor, cancer or malignancy comprises a solid cellular mass.

19. The method or use of claim 9, wherein the neoplasia, tumor, cancer or malignancy comprises hematopoietic cells.

20. The method or use of claim 9, wherein the neoplasia, tumor, cancer or malignancy comprises a carcinoma, sarcoma, lymphoma, leukemia, adenoma, adenocarcinoma, melanoma, glioma, glioblastoma, meningioma, neuroblastoma, retinoblastoma, astrocytoma, oligodendrocytoma, mesothelioma, reticuloendothelial, lymphatic or haematopoietic neoplasia, tumor, cancer or malignancy.

21. The method or use of claim 20, wherein the sarcoma comprises a lymphosarcoma, liposarcoma, osteosarcoma, chondrosarcoma, leiomyosarcoma, rhabdomyosarcoma or fibrosarcoma.

22. The method or use of claim 20, wherein the haematopoietic cell neoplasia, tumor, cancer or malignancy comprises a myeloma, lymphoma or leukemia.

23. The method or use of claim 9, wherein the neoplasia, tumor, cancer or malignancy comprises a metastatic melanoma.

24. The method or use of claim 9, wherein the neoplasia, tumor, cancer or malignancy comprises a lung, thyroid, head or neck, nasopharynx, throat, nose or sinuses, brain, spine, breast, adrenal gland, pituitary gland, thyroid, lymph, gastrointestinal (mouth, esophagus, stomach, duodenum, ileum, jejunum (small intestine), colon, rectum), genito-urinary tract (uterus, ovary, cervix, endometrial, bladder, testicle, penis, prostate), kidney, pancreas, liver, bone, bone marrow, lymph, blood, muscle, or skin, lung, biliary tract, or hematologic neoplasia, tumor, or cancer.

25. The method or use of any of claims 1-5, wherein the autoimmune disease or disorder comprises rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjogren's Syndrome, Crohn's disease, ulcerative colitis (UC), inflammatory bowel disease (IBD), aphthous ulcer, iritis, conjunctivitis, keratoconjunctivitis, cutaneous lupus erythematosus, scleroderma, uveitis posterior, autoimmune uveitis, allergic encephalomyelitis, aplastic anemia, pernicious anemia, pure red cell anemia, idiopathic thrombocytopenic purpura (ITP), polychondritis, Wegener's granulomatosis, Stevens-Johnson syndrome, idiopathic sprue, lichen planus, Graves' disease, sarcoidosis, primary biliary cirrhosis, Hashimoto's thyroiditis, autoimmune polyglandular syndrome, insulin-dependent diabetes mellitus (IDDM, type I diabetes), insulin-resistant diabetes mellitus (type 11 diabetes), autoimmune Addison's disease, pemphigus vulgaris, pemphigus foliaceus, dermatitis herpetiformis, autoimmune alopecia, vitiligo, autoimmune hemolytic anemia, Guillain-Barre syndrome, stiff-man syndrome, acute rheumatic fever, sympathetic ophthalmia, Goodpasture's syndrome, systemic necrotizing vasculitis, antiphospholipid syndrome, Behcet's disease, X-linked lymphoproliferative syndrome (SH2D1A/SAP deficiency), or Graft vs. Host Disease (GVHD).

26. The method or claim 5, wherein the infection comprises a bacterial, viral, fungal, or parasite infection.

27. The method or claim 5, wherein the infection comprises re-activation of latent viruses in the subject.

28. The method or use of any of claims 1-5, further comprising administering or use of a second, third or fourth autoimmune, anti-cancer or anti-tumor therapeutic agent.

29. The method or use of any of claims 1-5, wherein the humanin or humanin analog is administered or used prior to, substantially contemporaneously with or following administration of the autoimmune, anti-cancer or anti-tumor therapeutic agent.

30. The method or use of any of claims 1-5, wherein the humanin or humanin analog is administered or used in combination with the autoimmune, anti-cancer or anti-tumor therapeutic agent.

31. The method or use of any of claims 1-5, wherein the humanin or humanin analog is administered or used in one or more dose amounts of 0.05 to 50 mg/Kg per day.

32. The method or use of any of claims 1-5, wherein the humanin or humanin analog is administered or used in one or more dose amounts of 0.1 to 25 mg/Kg per day.

33. The method or use of any of claims 1-5, wherein the humanin or humanin analog is administered or used in one or more dose amounts of 0.5 to 15 mg/Kg per day.

34. The method or use of any of claims 1-5, wherein the humanin or humanin analog is administered or used in one or more dose amounts of 1.0 to 10 mg/Kg per day.

35. The method or use of any of claims 1-5, wherein the subject has undergone surgical resection, chemotherapy, immunotherapy, ionizing or chemical radiotherapy, local or regional thermal (hyperthermia) therapy, or vaccination.

36. The method or use of any of claims 1-5, wherein the subject is or is not a candidate for surgical resection, chemotherapy, immunotherapy, ionizing or chemical radiotherapy, local or regional thermal (hyperthermia) therapy, or vaccination.

37. The method or use of any of claims 1 to 36, wherein the subject is a mammal.

38. The method or use of any of claims 1 to 36, wherein the subject is a primate.

39. The method or use of any of claims 1 to 36, wherein the subject is a human.


SEQUENCE LISTING (94 sequences)

SEQ ID NO: 1 - protein, 24 aa, Artificial Sequence
  Description of Artificial Sequence Humanin Polypeptide
MAPRGFSCLLLLTSEIDLPVKRRA

SEQ ID NO: 2 - protein, 24 aa, Artificial Sequence
  Description of Artificial Sequence Humanin Polypeptide
MAPRGFSCLLLLTGEIDLPVKRRA

SEQ ID NO: 3 - protein, 24 aa, Artificial Sequence
  Description of Artificial Sequence Humanin Polypeptide
MAPRGFSCLLLLTXEIDLPVKRRA

SEQ ID NO: 4 - protein, 24 aa, Artificial Sequence
  Description of Artificial Sequence Humanin Polypeptide
MAPAGASCLLLLTGEIDLPVKRRA

SEQ ID NO: 5 - protein, 24 aa, Artificial Sequence
  Description of Artificial Sequence Humanin Polypeptide
MAPAGASCLLLLTXEIDLPVKRRA

SEQ ID NO: 6 - protein, 17 aa, Artificial Sequence
  Description of Artificial Sequence Humanin Polypeptide
PAGASCLLLLTXEIDLP

SEQ ID NO: 7 - protein, 17 aa, Artificial Sequence
  Description of Artificial Sequence Humanin Polypeptide
PAGASRLLLLTGEIDLP

SEQ ID NO: 8 - protein, 32 aa, Artificial Sequence
  Description of Artificial Sequence Humanin Polypeptide
EFLIVIKSMAPRGFSCLLLLTSEIDLPVKRRA

SEQ ID NO: 9 - protein, 32 aa, Artificial Sequence
  Description of Artificial Sequence Humanin Polypeptide
EFLIVIKSMAPRGFSCLLLLTGEIDLPVKRRA

SEQ ID NO: 10 - protein, 32 aa, Artificial Sequence
  Description of Artificial Sequence Humanin Polypeptide
EFLIVIKSMAPAGASCLLLLTGEIDLPVKRRA

SEQ ID NO: 11 - protein, 26 aa, Artificial Sequence
  Description of Artificial Sequence Humanin Polypeptide
SALLRSIPAPAGASRLLLLTGEIDLP

SEQ ID NO: 12 - protein, 24 aa, Artificial Sequence
  Description of Artificial Sequence Humanin Polypeptide
MAPRGFSCRLLLTSEIDLPVKRRA

SEQ ID NO: 13 - protein, 24 aa, Artificial Sequence
  Description of Artificial Sequence Humanin Polypeptide
MTPRGFSCLLLPTSETDLPVKRRX

SEQ ID NO: 14 - protein, 24 aa, Artificial Sequence
  Description of Artificial Sequence Humanin Polypeptide
MAPRGFSCLLLSTSEIDLPVKRXX

SEQ ID NO: 15 - protein, 24 aa, Artificial Sequence
  Description of Artificial Sequence Humanin Polypeptide
MAPRGFSCLLLSTSEIDLPVKRRA

SEQ ID NO: 16 - protein, 24 aa, Artificial Sequence
  Description of Artificial Sequence Humanin Polypeptide
MCHWAGGASNTGDARGDVFGKQAG

SEQ ID NO: 17 - protein, 26 aa, Artificial Sequence
  Description of Artificial Sequence Humanin Polypeptide
MGVKFFTLSTRFFPSVQRAVPLWTNS

SEQ ID NO: 18 - protein, 38 aa, Artificial Sequence
  Description of Artificial Sequence Humanin Polypeptide
MLGYNFSSFPCGTISIAPGFNFYRLYFTWVNGLAKVVW

SEQ ID NO: 19 - protein, 26 aa, Artificial Sequence
  Description of Artificial Sequence Humanin Polypeptide
MLEVMFLVNRRGKICRVPFTFFNLSL

SEQ ID NO: 20 - protein, 24 aa, Artificial Sequence
  Description of Artificial Sequence Humanin Polypeptide
MYCSEVGFCSEVAPTEIFNAGLVV

SEQ ID NO: 21 - protein, 20 aa, Artificial Sequence
  Description of Artificial Sequence Humanin Polypeptide
MLDQDIPMVQPLIKVRLFND

SEQ ID NO: 22 - protein, 24 aa, Artificial Sequence
  Description of Artificial Sequence Humanin Polypeptide
MAPRGASCLLLLTSEIDLPVKRRA

SEQ ID NO: 23 - protein, 24 aa, Artificial Sequence
  Description of Artificial Sequence Humanin Polypeptide
MAPRGASCLLLLTGEIDLPVKRRA

SEQ ID NO: 24 - protein, 24 aa, Artificial Sequence
  Description of Artificial Sequence Humanin Polypeptide
MAPRGFACLLLLTSEIDLPVKRRA

SEQ ID NO: 25 - protein, 24 aa, Artificial Sequence
  Description of Artificial Sequence Humanin Polypeptide
MAPRGFSPLLLLTSEIDLPVKRRA

SEQ ID NO: 26 - protein, 24 aa, Artificial Sequence
  Description of Artificial Sequence Humanin Polypeptide
MAPRGFSCLLLLTXEIDLPVKRRA

SEQ ID NO: 27 - protein, 24 aa, Artificial Sequence
  Description of Artificial Sequence Humanin Polypeptide
MAPRGFXCLLLLTSEIDLPVKRRA

SEQ ID NO: 28 - protein, 24 aa, Artificial Sequence
  Description of Artificial Sequence Humanin Polypeptide
MAPRGFXCLLLLTXEIDLPVKRRA

SEQ ID NO: 29 - protein, 24 aa, Artificial Sequence
  Description of Artificial Sequence Humanin Polypeptide
MAPRGFSCLLLLTXEIDLPVKRRA

SEQ ID NO: 30 - protein, 24 aa, Artificial Sequence
  Description of Artificial Sequence Humanin Polypeptide
MAPRGFXCLLLLTSEIDLPVKRRA

SEQ ID NO: 31 - protein, 24 aa, Artificial Sequence
  Description of Artificial Sequence Humanin Polypeptide
MAPRGFXCLLLLTXEIDLPVKRRA

SEQ ID NO: 32 - protein, 24 aa, Artificial Sequence
  Description of Artificial Sequence Humanin Polypeptide
MAPAGASCLLLLTXEIDLPVKRRA

SEQ ID NO: 33 - protein, 17 aa, Artificial Sequence
  Description of Artificial Sequence Humanin Polypeptide
PAGASCLLLLTXEIDLP

SEQ ID NO: 34 - protein, 32 aa, Artificial Sequence
  Description of Artificial Sequence Humanin Polypeptide
EFLIVIKSMAPRGFACLLLLTSEIDLPVKRRA

SEQ ID NO: 35 - protein, 32 aa, Artificial Sequence
  Description of Artificial Sequence Humanin Polypeptide
EFLIVIKSMAPRGFSCLLLLTGEIDLPVKKKK

SEQ ID NO: 36 - protein, 32 aa, Artificial Sequence; identical to SEQ ID NO: 8
  Description of Artificial Sequence Humanin Polypeptide

SEQ ID NO: 37 - protein, 32 aa, Artificial Sequence
  Description of Artificial Sequence Humanin Polypeptide
EFLIVIKSMAPRGFSALLLLTSEIDLPVKRRA

SEQ ID NO: 38 - protein, 32 aa, Artificial Sequence; identical to SEQ ID NO: 9
  Description of Artificial Sequence Humanin Polypeptide

SEQ ID NO: 39 - protein, 32 aa, Artificial Sequence; identical to SEQ ID NO: 10
  Description of Artificial Sequence Humanin Polypeptide

SEQ ID NO: 40 - protein, 24 aa, Artificial Sequence
  Description of Artificial Sequence Humanin Polypeptide
MAPRGFSCLLLLTSEIDLPVKRRA

SEQ ID NO: 41 - protein, 24 aa, Artificial Sequence
  Description of Artificial Sequence Humanin Polypeptide
MAPRGFSCLLLLTGEIDLPVKRRA

SEQ ID NO: 42 - protein, 24 aa, Artificial Sequence
  Description of Artificial Sequence Humanin Polypeptide
MAPRGFSALLLLTSEIDLPVKRRA

SEQ ID NO: 43 - protein, 24 aa, Artificial Sequence
  Description of Artificial Sequence Humanin Polypeptide
MAPRGFSCLLLLTXEIDLPVKRRA

SEQ ID NO: 44 - protein, 24 aa, Artificial Sequence
  Description of Artificial Sequence Humanin Polypeptide
MAPRGFXCLLLLTSEIDLPVKRRA

SEQ ID NO: 45 - protein, 24 aa, Artificial Sequence
  Description of Artificial Sequence Humanin Polypeptide
MAPRGFXCLLLLTXEIDLPVKRRA

SEQ ID NO: 46 - protein, 24 aa, Artificial Sequence
  Description of Artificial Sequence Humanin Polypeptide
MAPRGFSCLLLLTXEIDLPVKRRA

SEQ ID NO: 47 - protein, 24 aa, Artificial Sequence
  Description of Artificial Sequence Humanin Polypeptide
MAPRGFXCLLLLTSEIDLPVKRRA

SEQ ID NO: 48 - protein, 24 aa, Artificial Sequence
  Description of Artificial Sequence Humanin Polypeptide
MAPRGFXCLLLLTXEIDPPVKRRA

SEQ ID NO: 49 - protein, 24 aa, Artificial Sequence
  Description of Artificial Sequence Humanin Polypeptide
MAPAGASCLLLLTXEIDLPVKRRA

SEQ ID NO: 50 - protein, 17 aa, Artificial Sequence
  Description of Artificial Sequence Humanin Polypeptide
PAGASCLLLLTXEIDLP

SEQ ID NO: 51 - protein, 24 aa, Artificial Sequence
  Description of Artificial Sequence Humanin Polypeptide
MAPRGFACLLLLTSEIDLPVKRRA

SEQ ID NO: 52 - protein, 17 aa, Artificial Sequence
  Description of Artificial Sequence Humanin Polypeptide
PRGFACLLLLTSEIDLP

SEQ ID NO: 53 - protein, 25 aa, Artificial Sequence
  Description of Artificial Sequence Humanin Polypeptide
YMAPRGFSCLLLLTGEIDLPVKKKK

SEQ ID NO: 54 - protein, 32 aa, Artificial Sequence; identical to SEQ ID NO: 34
  Description of Artificial Sequence Humanin Polypeptide

SEQ ID NO: 55 - protein, 32 aa, Artificial Sequence; identical to SEQ ID NO: 35
  Description of Artificial Sequence Humanin Polypeptide

SEQ ID NO: 56 - protein, 32 aa, Artificial Sequence; identical to SEQ ID NO: 8
  Description of Artificial Sequence Humanin Polypeptide

SEQ ID NO: 57 - protein, 32 aa, Artificial Sequence; identical to SEQ ID NO: 37
  Description of Artificial Sequence Humanin Polypeptide

SEQ ID NO: 58 - protein, 32 aa, Artificial Sequence; identical to SEQ ID NO: 9
  Description of Artificial Sequence Humanin Polypeptide

SEQ ID NO: 59 - protein, 8 aa, Artificial Sequence
  Description of Artificial Sequence Humanin Polypeptide
EFLIVIKS

SEQ ID NO: 60 - protein, 24 aa, Artificial Sequence
  Description of Artificial Sequence Humanin Polypeptide
MAPAGASCLLLLTGEIDLPVKRRA

SEQ ID NO: 61 - protein, 32 aa, Artificial Sequence; identical to SEQ ID NO: 10
  Description of Artificial Sequence Humanin Polypeptide

SEQ ID NO: 62 - protein, 17 aa, Artificial Sequence
  Description of Artificial Sequence Humanin Polypeptide
PRGFSCLLLLTGEIDLP

SEQ ID NO: 63 - protein, 4 aa, Artificial Sequence
  Description of Artificial Sequence Humanin Polypeptide Residues
RGFS

SEQ ID NO: 64 - protein, 4 aa, Artificial Sequence
  Description of Artificial Sequence Humanin Polypeptide Residues
AGFS

SEQ ID NO: 65 - protein, 4 aa, Artificial Sequence
  Description of Artificial Sequence Humanin Polypeptide Residues
RAFS

SEQ ID NO: 66 - protein, 4 aa, Artificial Sequence
  Description of Artificial Sequence Humanin Polypeptide Residues
RGAS

SEQ ID NO: 67 - protein, 4 aa, Artificial Sequence
  Description of Artificial Sequence Humanin Polypeptide Residues
RGFA

SEQ ID NO: 68 - protein, 4 aa, Artificial Sequence
  Description of Artificial Sequence Humanin Polypeptide Residues
RGAA

SEQ ID NO: 69 - protein, 4 aa, Artificial Sequence
  Description of Artificial Sequence Humanin Polypeptide Residues
RAFA

SEQ ID NO: 70 - protein, 4 aa, Artificial Sequence
  Description of Artificial Sequence Humanin Polypeptide Residues
RAAS

SEQ ID NO: 71 - protein, 4 aa, Artificial Sequence
  Description of Artificial Sequence Humanin Polypeptide Residues
RAAA

SEQ ID NO: 72 - protein, 4 aa, Artificial Sequence
  Description of Artificial Sequence Humanin Polypeptide Residues
AGFA

SEQ ID NO: 73 - protein, 4 aa, Artificial Sequence
  Description of Artificial Sequence Humanin Polypeptide Residues
AGAS

SEQ ID NO: 74 - protein, 4 aa, Artificial Sequence
  Description of Artificial Sequence Humanin Polypeptide Residues
AGAA

SEQ ID NO: 75 - protein, 4 aa, Artificial Sequence
  Description of Artificial Sequence Humanin Polypeptide Residues
AAFS

SEQ ID NO: 76 - protein, 4 aa, Artificial Sequence
  Description of Artificial Sequence Humanin Polypeptide Residues
AAFA

SEQ ID NO: 77 - protein, 4 aa, Artificial Sequence
  Description of Artificial Sequence Humanin Polypeptide Residues
AAAS

SEQ ID NO: 78 - protein, 4 aa, Artificial Sequence
  Description of Artificial Sequence Humanin Polypeptide Residues
AAAA

SEQ ID NO: 79 - protein, 4 aa, Artificial Sequence
  Description of Artificial Sequence Humanin Polypeptide Residues
EIDL

SEQ ID NO: 80 - protein, 4 aa, Artificial Sequence
  Description of Artificial Sequence Humanin Polypeptide Residues
AIDL

SEQ ID NO: 81 - protein, 4 aa, Artificial Sequence
  Description of Artificial Sequence Humanin Polypeptide Residues
EADL

SEQ ID NO: 82 - protein, 4 aa, Artificial Sequence
  Description of Artificial Sequence Humanin Polypeptide Residues
EIAL

SEQ ID NO: 83 - protein, 4 aa, Artificial Sequence
  Description of Artificial Sequence Humanin Polypeptide Residues
EIDA

SEQ ID NO: 84 - protein, 4 aa, Artificial Sequence
  Description of Artificial Sequence Humanin Polypeptide Residues
EIAA

SEQ ID NO: 85 - protein, 4 aa, Artificial Sequence
  Description of Artificial Sequence Humanin Polypeptide Residues
EADA

SEQ ID NO: 86 - protein, 4 aa, Artificial Sequence
  Description of Artificial Sequence Humanin Polypeptide Residues
EAAL

SEQ ID NO: 87 - protein, 4 aa, Artificial Sequence
  Description of Artificial Sequence Humanin Polypeptide Residues
EAAA

SEQ ID NO: 88 - protein, 4 aa, Artificial Sequence
  Description of Artificial Sequence Humanin Polypeptide Residues
AIDA

SEQ ID NO: 89 - protein, 4 aa, Artificial Sequence
  Description of Artificial Sequence Humanin Polypeptide Residues
AIAL

SEQ ID NO: 90 - protein, 4 aa, Artificial Sequence
  Description of Artificial Sequence Humanin Polypeptide Residues
AIAA

SEQ ID NO: 91 - protein, 4 aa, Artificial Sequence
  Description of Artificial Sequence Humanin Polypeptide Residues
AADL

SEQ ID NO: 92 - protein, 4 aa, Artificial Sequence
  Description of Artificial Sequence Humanin Polypeptide Residues
AADA

SEQ ID NO: 93 - protein, 4 aa, Artificial Sequence
  Description of Artificial Sequence Humanin Polypeptide Residues
AAAL

SEQ ID NO: 94 - protein, 4 aa, Artificial Sequence
  Description of Artificial Sequence Humanin Polypeptide Residues
AAAA